(19) United States
(12) Patent Application Publication
(10) Pub. No.: US 2022/0331415 A1
(43) Pub. Date: Oct. 20, 2022

(54) IMMUNOACTIVE MICROPARTICLES AND USES THEREOF

(71) Applicant: The Regents of the University of California, Oakland, CA (US)

(72) Inventors: Manish J. BUTTE, Los Angeles, CA (US); Mohammad Mahdi HASANI-SADRABADI, Los Angeles, CA (US); Fatemeh S. MAJEDI, Los Angeles, CA (US); Song LI, Beverly Hills, CA (US)

(73) Assignee: The Regents of the University of California, Oakland, CA (US)

(21) Appl. No.: 17/642,515

(22) PCT Filed: Sep. 18, 2020
(86) PCT No.: PCT/US20/51419

§ 371 Date: Mar. 11, 2022

Related U.S. Application Data
(60) Provisional application No. 62/902,376, filed on Sep. 18, 2019.

(51) Int. Cl. A61K 39/00; A61K 9/16; A61K 31/727; A61K 39/39; A61K 31/4709; A61K 31/4439; A61K 9/14; A61K 45/06; A61P 35/00; A61P 37/04; A61P 7/02; A61P 37/06; A61P 31/00; A61P 37/08; A61K 47/02

(52) U.S. Cl. CPC A61K 39/0011; A61K 39/0008; A61K 9/1652; A61K 31/727; A61K 39/39; A61K 31/4709; A61K 31/4439; A61K 9/14; A61K 9/1694; A61K 45/06; A61P 35/00; A61P 37/04; A61P 7/02; A61P 37/06; A61K 39/001; A61P 31/00; A61P 37/08; A61K 47/02; A61K 2039/55533; A61K 2039/55538; A61K 2039/55527; A61K 2039/55522; A61K 2039/55516; A61K 2039/5158; A61K 2039/55561

(57) ABSTRACT

A microparticle is described comprising an antigen and a costimulatory component derived from an antigen presenting cell. The microparticle may be used for stimulating T cells ex vivo, followed by administration to a subject, e.g., as part of a personalized, customized therapeutic treatment of cancer or a tumor, an autoimmune disease or an allergic reaction, hypersensitivity reaction, an infection or infectious disease, an injury or other damage, a transplant or other surgical site, or a blood clot. It may also be used for the controlled release of a cytokine for the regulation of immunity in general and for other therapeutic uses. Methods of treating a disease or medical condition in a subject by exposing leukocytes from the subject to the microparticle, then reinfusing the leukocytes into the subject are provided. Methods of preparing an activated cytotoxic T cell population specific for an antigen are also provided.

### CROSS-REFERENCE TO RELATED APPLICATIONS

This application claims priority to U.S. Provisional Patent Application 62/902,376, filed Sep. 18, 2019, which is incorporated by reference herein in its entirety.

### STATEMENT OF GOVERNMENT INTEREST

The invention described herein was supported in whole or in part by grants from The National Institutes of Health (Grant Nos. R01 GM110482 and 1R56DE029157). The government has certain rights in the invention. In addition, interleukin-2 used for this study was provided by the BRB Preclinical Repository of the National Cancer Institute, Frederick, Md., USA.

### FIELD OF INTEREST

This disclosure relates to microparticles comprising an antigen and a costimulatory component derived from an antigen presenting cell. These microparticles may be used for stimulating T cells ex vivo, followed by administration to a subject, e.g., as part of a personalized, customized therapeutic treatment of cancer or a tumor, an autoimmune disease or an allergic reaction, hypersensitivity reaction, an infection or infectious disease, an injury or other damage, a transplant or other surgical site, or a blood clot. They may also be used for the controlled release of a cytokine for the regulation of immunity in general and for other therapeutic uses.

### BACKGROUND

Activating large numbers of endogenous T cells to attack tumor neoantigens is conceptually straightforward and serves as the basis for a number of efforts in cancer vaccines. Identifying the neoantigen genetically or computationally is slow and has considerable hurdles to achieve real-time treatment.

Therapeutic modulation of immunity has made significant headway in the fight against cancer. However, global immunomodulation results in systemic adverse effects including severe inflammation, provocation of autoimmunity, and susceptibility to infection. Cytokines influence the proliferation and differentiation of cultured, primary T cells. Augmentation and engineering of immune responses have major applications in combating cancers, including solid tumor cancers.

Activation of cytotoxic T cells for cancer immunotherapy has significant potential for patients with tumors or following tumor resection, but obstacles persist in available procedures. Transforming growth factor-beta (TGFβ) is a potent component of the tumor microenvironment, which promotes cancer growth and metastasis and promotes the induction of regulatory T cells (Tregs; T regulatory cells) from the helper T cells drawn to the tumor. TGF-β also potently inhibits cytotoxic T cells in the tumor microenvironment. TGF-β has, therefore, become a target in the enhancement of immunotherapy. However, systemic TGF-β inhibition in preclinical models has shown major adverse effects on the cardiovascular, gastrointestinal, and skeletal systems, owing to the pleotropic effects that TGF-β plays across the body. Similarly, IL-2 is a cytokine that plays the major role in activation and expansion of helper and cytotoxic T cells (CTLs) to fight infections and cancer. IL-2 also helps activate natural killer cells for fighting viruses and cancer. Unfortunately, systemic delivery of IL-2 has been shown to be inefficient and has additional limitations including continuous secretion eliciting non-specific immune response.

A tumor, whether benign or malignant, is caused by abnormal growth of cells or a tissue. Cancer is an abnormal and malignant state in which uncontrolled proliferation of one or more cell populations interferes with normal biological functioning. Standard treatments for cancer include surgery, chemotherapy, and radiation therapy. T cell immunotherapy is a promising approach for cancer. However, significant challenges hamper its therapeutic potential, including insufficient activation, delivery, and clonal expansion of T cells into the tumor environment. Even non-cancerous tumors may pose significant health challenges, such as when they are located at treatment site that is difficult to access or when they chronically recur.

Some infectious and non-infectious medical conditions exist, at least initially, in localized environments within the body. For example, these types of diseases and conditions are often difficult to treat without systemic exposure to therapeutic agents, which may have significant side effects. Some autoimmune diseases (e.g., rheumatoid arthritis, juvenile dermatomyositis, psoriasis, psoriatic arthritis, sarcoidosis, lupus, Crohn's disease, eczema, vasculitis, ulcerative colitis, multiple sclerosis) may present with at least some localized symptoms or symptoms in a particular system of the body, but treatment options may leave the patient having to choose between alleviating one or more symptoms (e.g., use of a non-steroidal anti-inflammatory drug [NSAID] or an antihistamine or a dermatological ointment or cream providing limited relief of a given symptom) or systemic exposure of the entire body to a more aggressive treatment (e.g., methotrexate) with a concomitant increase in potentially dangerous side effects. Likewise, some infectious diseases (e.g., shingles) or initially localized infections (e.g., methicillin-resistant Staphylococcus aureus [MRSA] infection) may have few treatment options or may require the use of more aggressive systemic treatments. In addition, traumatic injury, chronic damage (e.g., osteoarthritis, type 1 diabetes, rheumatoid arthritis, lupus), surgery, or a blood clot may necessitate the use of more aggressive systemic treatments, notwithstanding the limited location of the injury or surgical site. Furthermore, the concern over potential rejection of a transplant (e.g., a transplanted organ) necessitates aggressive systemic treatments with immune suppression drugs, often with significant side effects, also notwithstanding the limited location of the transplant site.

Thus, there remains an unmet need for compositions and methods of treatment of cancers and other tumors, for example, but not limited to, treatment of benign or malignant solid tumors. A major gap in treatment exists, wherein there is an inability to target and concentrate factors where most needed in the treatment of solid tumors, while avoiding systemic exposure to immunomodulatory agents.

Similarly, there remains an unmet need for compositions and methods of treatment of, for example, but not limited to, infectious and non-infectious medical conditions, injuries, damage, surgery, and transplant. A major gap in treatment exists, wherein there is an inability to target and concentrate factors and other treatments where most needed in the personalized, customized, or targeted treatment of symptoms, including localized symptoms, while avoiding systemic exposure to immunomodulatory agents.

Accordingly, there is a need for improving the effectiveness of immunotherapy.

### SUMMARY

Provided herein is a microparticle effective on a broad range of cell membranes, including, but not limited to, membranes of tumor cells, stem and progenitor cells, immune cells (e.g., B cells, T cells, natural killer cells, and macrophage cells), monocytes (leukocytes), lymphocytes, erythrocytes (red blood cells), and platelets (thrombocytes).

In some aspects, provided herein is a microparticle comprising: an antigen; and a costimulatory component derived from an antigen presenting cell. In some embodiments, the antigen comprises a tumor antigen; a cancer-specific antigen; a self antigen; an IgE receptor-specific antigen; a pathogen-specific antigen; an antigen specific to an organ, tissue, or cell of interest; an antigen specific to a transplanted organ, tissue, or cell of interest; a macrophage-specific antigen; an erythrocyte-specific antigen; a platelet-specific antigen; a platelet factor 5-specific antigen; a fibrinogen-specific antigen; a stem cell- or progenitor cell-specific antigen; a lymphocyte-specific antigen; a monocyte-specific antigen; an immune cell-specific antigen; or a stromal cell-specific antigen; or a combination thereof.

In some embodiments, the antigen comprises a tumor antigen comprising a membrane isolated from a tumor cell or a tumor tissue; or the antigen comprises a cancer-specific antigen comprising a membrane isolated from a cancer cell or a cancer tissue. In other embodiments, the antigen comprises a self antigen comprising a membrane isolated from a cell, a tissue, or an organ targeted by an autoimmune disease or undergoing autoimmune attack; the antigen comprises an IgE receptor-specific antigen comprising a membrane isolated from a mast cell or a basophil in response to an allergic reaction or hypersensitivity reaction; the antigen comprises a pathogen-specific antigen comprising a membrane isolated from a cell of a pathogen or a viral envelope or a viral capsid; the antigen comprises a macrophage-specific antigen or an immune cell (e.g., a B cell, a T cell, a natural killer [NK] cell, or a macrophage)-specific antigen comprising a membrane isolated from a macrophage or immune cell (e.g., a B cell, a T cell, a natural killer [NK] cell, or a macrophage, respectively, of interest) of interest; the antigen comprises an antigen specific to an organ, tissue, or cell of interest comprising a membrane isolated from the organ, tissue, or cell of interest; the antigen comprises an antigen specific to a transplanted organ, tissue, or cell of interest comprising a membrane isolated from the organ, tissue, or cell of interest; or the antigen comprises an erythrocyte-specific antigen, a platelet-specific antigen, a platelet factor 5-specific antigen, a fibrinogen-specific antigen comprising a membrane isolated from an erythrocyte or a platelet (e.g., a thrombocyte); the antigen comprises a stem cell- or progenitor cell-specific antigen comprising a membrane isolated from a stem cell or a progenitor cell, respectively, of interest; a lymphocyte-specific antigen comprising a membrane isolated from a lymphocyte of interest; a monocyte-specific antigen comprising a membrane isolated from a monocyte (e.g., a leukocyte) of interest; or a stromal cell-specific antigen comprising a membrane isolated from a stromal cell. In some embodiments, the antigen comprises a B cell-specific antigen comprising a membrane isolated from a B cell or wherein the antigen comprises a T cell-specific antigen comprising a membrane isolated from a T cell.

In some embodiments, the tumor cell or tumor tissue is obtained from a surgically obtained tumor, tumor biopsy, or tumor sample. In some embodiments, the cell, tissue, or organ of interest is obtained from a surgically obtained cell, tissue, or organ of interest, a biopsy, or a sample. In some embodiments, the antigen is obtained from a cell grown in vitro or a culture media thereof.

In some embodiments, the costimulatory component from an antigen presenting cell comprises a membrane isolated from an antigen presenting cell. In some embodiments, the antigen presenting cell is stimulated in vitro before the membrane is isolated.

In some embodiments, the microparticle comprises a polymer. In some embodiments, the polymer is a biocompatible polymer. In some embodiments, the polymer is alginate, hyaluronic acid, or chitosan. In some embodiments, the microparticle further comprises heparin. In other embodiments, the microparticle comprises alginate and heparin. In some embodiments, the polymer is cross-linked. In some embodiments, the microparticle further comprises paramagnetic nanoparticles (e.g., superparamagnetic iron oxide nanoparticles [SPIONs]).

In some embodiments, the microparticle further comprises at least one immunoregulatory compounds. In some embodiments, the at least one immunoregulatory compound comprises an immunostimulatory compound. In other embodiments, the at least one immunoregulatory compound comprises an immunosuppression compound. In some embodiments, the at least one immunoregulatory compound comprises a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid. In some embodiments, the cytokine comprises an interleukin (IL). In some embodiments, the cytokine comprises interleukin-2 (IL-2), interleukin-4 (IL-4), interleukin-6 (IL-6), interleukin-7 (IL-7), interleukin-10 (IL-10), interleukin-12 (IL-12), or interleukin-15 (IL-15). In some embodiments, the interleukin is an IL-2 superkine. In some embodiments, the IL-2 superkine comprises the sequence as set forth in SEQ ID NO: 3. In some embodiments, the chemokine comprises stromal cell-derived factor 1a (SDF-1a). In some embodiments, the growth factor comprises transforming growth factor-beta (TGF-β), vascular endothelial growth factor (VEGF), or bone morphogenetic protein-2 (BMP-2). In some embodiments, the microparticle comprises IL-2 and TGF-β.

In some embodiments, the microparticle further comprises at least one compound that regulates induction of regulatory T cells (Tregs). In some embodiments, the at least one compound that regulates induction of Tregs comprises a compound that suppresses induction of Tregs. In some embodiments, the compound that suppresses induction of Tregs comprises a TGF-β inhibitor. In some embodiments, the TGF-β inhibitor is a TGF-β receptor inhibitor. In some embodiments, the TGF-β inhibitor is galinusertib (LY2157299) or SB505124. In other embodiments, the at least one compound that regulates induction of Tregs comprises a compound that induces Tregs. In some embodiments, the compound that induces Tregs is a TGF-β or an activator thereof.

In some embodiments, the microparticle further comprises a small molecule. In some embodiments, the microparticle further comprises a hydrophobic therapeutic agent.

In some aspects, provided herein is a nanoparticle comprising: (a) an antigen specific to a cell or virus of interest; and (b) a costimulatory component derived from an antigen presenting cell.

In some aspects, provided herein is a method for preparing an immunoactive microparticle or an immunoactive nanoparticle, the method comprising: providing a biocompatible polymer; combining said biocompatible polymer in a microfluidic droplet generator with a crosslinker to form the microparticle or the nanoparticle; incubating the microparticle or the nanoparticle with: (i) an antigen specific to a cell or virus of interest; and (ii) a costimulatory component derived from an antigen presenting cell.

In some aspects, provided herein is a method for preparing an activated cytotoxic T cell population specific for an antigen of interest comprising: (a) providing a microparticle or a nanoparticle comprising: (i) an antigen specific to a cell or virus of interest; and (ii) a costimulatory component derived from an antigen presenting cell; (b) obtaining leukocytes from a subject; and (c) exposing the leukocytes in vitro or ex vivo to the microparticle or the nanoparticle.

In some aspects, provided herein is a method for treating a disease or medical condition, or of alleviating symptoms thereof, at a focus of interest in a subject in need, said method comprising: (a) providing a microparticle or a nanoparticle comprising: (i) an antigen specific to a cell or virus of interest; and (ii) a costimulatory component derived from an antigen presenting cell; (b) obtaining a leukocyte from the subject; (c) exposing the leukocyte to the microparticle or the nanoparticle; and (d) infusing the leukocytes into the subject.

In some embodiments, the disease or medical condition comprises a tumor, a suspected tumor, or a resected tumor. In some embodiments, the disease or medical condition comprises an autoimmune disease; the disease or medical condition comprises an allergic reaction or hypersensitivity reaction, the disease or medical condition comprises a localized infection or an infectious disease; the disease or medical condition comprises an injury or a site of chronic damage; the disease or medical condition comprises a surgical site; the disease or medical condition comprises a transplanted organ, tissue, or cell; or the disease or medical condition comprises a blood clot causing or at risk for causing a myocardial infarction, an ischemic stroke, or a pulmonary embolism.

In some embodiments, the antigen comprises a tumor antigen; and the microparticle further comprises at least one immunoregulatory compound comprising a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid. In some embodiments, the cytokine comprises interleukin-4 (IL-4), interleukin-6 (IL-6), interleukin-7 (IL-7), interleukin-10 (IL-10), interleukin-12 (IL-12), interleukin-15 (IL-15), or an IL-2 superkine. the chemokine comprises SDF-1a; and the growth factor comprises TGF-β, VEGF, or BMP-2. In some embodiments, the microparticle further comprises a TGF-β inhibitor. In some embodiments, the tumor antigen is specific for a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

In some embodiments, said treating reduces the size of the tumor, eliminates the tumor, slows the growth or regrowth of the tumor, or prolongs survival of said subject or any combination thereof; said treating reduces or eliminates inflammation or another symptom of said autoimmune-targeted or symptomatic focus of said autoimmune disease, prolongs survival of said subject, or any combination thereof; reduces or eliminates inflammation or another symptom of allergic reaction or hypersensitivity reaction at said reactive focus of said allergic reaction or hypersensitivity reaction, prolongs survival of said subject, or any combination thereof; reduces or eliminates infection or symptoms at said focus of infection or symptoms of said localized infection or infectious disease, prolongs survival of said subject, or any combination thereof; reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof; reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof; reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof.

In some embodiments, at the site, T cells are stimulated to target the antigen, and the induction of Tregs is suppressed. In other embodiments, at the site, T cells are suppressed from targeting the antigen, and Tregs are induced.

In some embodiments, the antigen comprises a self antigen; and the microparticle further comprises at least one immunoregulatory compound comprising a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid. In some embodiments, the cytokine comprises interleukin-4 (IL-4), interleukin-6 (IL-6), interleukin-7 (IL-7), interleukin-10 (IL-10), interleukin-12 (IL-12), or interleukin-15 (IL-15), or an IL-2 superkine; the chemokine comprises SDF-1a; and the growth factor comprises TGF-β, VEGF, or BMP-2. In some embodiments, the microparticle further comprises IL-2, TGF-β, or a TGF-β activator.

In other aspects, provided herein is a method for treating a disease or medical condition, or of alleviating symptoms thereof, at a focus of interest in a subject in need, said method comprising: providing a microparticle comprising an antigen specific to a cell or virus of interest and a costimulatory component derived from an antigen presenting cell; obtaining a leukocyte from the subject; exposing the leukocyte to the microparticle; and infusing the leukocytes into the subject. In some embodiments, the antigen is obtained from a sample from the subject. In some embodiments, the leukocyte is obtained from whole blood or pheresis. In some embodiments, the microparticle further comprises paramagnetic nanoparticles and is separated from the leukocyte by magnetic separation. In some embodiments, the subject is treated with at least one other immunotherapy. In some embodiments, the disease or medical condition comprises a tumor or a cancer, the antigen comprises a tumor antigen or a cancer antigen, and the subject is treated with at least one other anti-tumor or anti-cancer therapy. In some embodiments, the disease or medical condition comprises an autoimmune disease, the antigen comprises a self antigen, and the subject is treated with at least one other immunotherapy.

In still other aspects, provided herein is a method for preparing an activated cytotoxic T cell population specific for an antigen of interest comprising: providing a microparticle comprising an antigen specific to a cell or virus of interest and a costimulatory component derived from an antigen presenting cell; obtaining leukocytes from a subject; and exposing the leukocytes in vitro or ex vivo to the microparticle. In some embodiments, the leukocytes are obtained from whole blood or pheresis. In some embodiments, the microparticle further comprises paramagnetic nanoparticles and is separated from the leukocytes by magnetic separation. In some embodiments, the subject is treated with at least one other immunotherapy.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B depict the formation of microparticles of the invention (A) and their use in activating T cells (B). FIG. 1A is a schematic depicting a non-limiting example of a method of making artificial antigen presenting cells (aAPCs) that promote T cell responses to tumors by combining the membranes of tumor cells and the membranes of activated antigen presenting cells (APCs) in a microparticle. FIG. 1B depicts the subsequent use of an exemplary microparticle in activating a primary T cell ex vivo.

FIG. 2 depicts fabrication of the microparticles of the invention. In a non-limiting example, microparticles were generated by microfluidic droplet generation using alginate-heparin as the main component (left). The alginate was crosslinked using a 4-arm PEG hydrazide crosslinker. Embedded in each microparticle were superparamagnetic nanoparticles as well. The flow ratio in the generator can be adjusted for different droplet diameters which translate to particle sizes (and dispersities), as shown in the graph (top right). The size distribution of the resulting microparticles is also shown (bottom right).

FIG. 3 depicts the mechanical properties of the microparticles. Graphs show the load vs. indentation relationships (left), and Young's modulus (right), of a hard and soft particle.

FIG. 4 depicts the affinity of microparticles with and without heparin for IL-2. Graphs compare the binding affinity vs. time (left) and the dissociation constants (right).

FIG. 5 shows a series of graphs demonstrating the binding and release of IL-2 by microparticles. IL-2 binding efficiency to soft and hard microparticles is shown (left), which is not dependent on the microparticle size (center). The release of IL-2 over time of the different microparticles is shown (right).

FIG. 6 shows the size of microparticles comprising magnetic nanoparticles. An exemplary alginate-heparin microparticle comprising magnetic nanoparticles (100 nm, —COOH) (left). A graph shows the distribution by number of magnetic nanoparticles per microparticle (right).

FIG. 7 is a series of confocal laser scanning micrographs (CLSMs) showing the relative sizes of B cells based on their surface, 4G10 (red), and cytoplasmic, actin (green), markers, as well as nuclei (DAPI: blue) which demonstrating that B cells have large nuclei.

FIG. 8 shows that microparticles have been coated with APC membrane components. Flow cytometry was conducted on the B cells. As shown in the graphs (top), they have plentiful expression of the cell-surface adhesion molecule ICAM-1, the antigen presenting molecule MHC-II, and the costimulatory ligand B7-1 (CD80). The schematic image (bottom right) depicts B cell membrane coated microparticles. The CLSM (bottom center) shows B cell membrane coated microparticles. As shown in the graph (bottom right), when the sizes of the particles were normalized to the size of the B cells, it was observed that expression of these three markers was exactly comparable to the B cells themselves.

FIG. 9 shows a series of bright field images depicting in vitro activation of T cells by different size microparticles. Three different sizes (center) of microparticles were prepared as both hard and soft microparticles (right). Bigger cell aggregates (dark parts) demonstrating higher level of T cell activation and expansion.

FIG. 10 shows the method for measurement of proliferation using carboxyfluorescein succinimidyl ester (CFSE) is a fluorescent cell staining dye (CFSE). CFSE is staining the mother cells and the T cell proliferation can be tracked due to the progressive halving of CFSE fluorescence within daughter cells following each cell division.

FIG. 11 shows a series of graphs comparing proliferation (proliferation indices, left; percentages of proliferated cells, right) of cultured T cells by soft and hard microparticles of different sizes. Larger particles with stiffer mechanical properties can promote higher level of cellular activation and as a result higher proliferation can be achieved.

FIG. 12 depicts immune synapse formation by soft and hard microparticles of different sizes. As shown in the graphs (left), the three sizes of microparticles formed immune synapses, but higher synapse volumes were achieved with the hard microparticles. The CLSMs (right) show accommodation of actin (red) and LFA-1 (green) at the site of immune synapse. More activation resulted in larger immune synapse volume.

FIG. 13 depicts the formation of Tregs by TGF-β release from microparticles (top). Graphs show the dissociation constant (left) and TGF-β binding efficiency (center) of alginate vs. alginate-heparin microparticles, as well as the cumulative release of TGF-β (right). dissociation constant was measured using surface plasmon resonance (SPR) technique to evaluate enhanced affinity of TGF-β toward the microparticles in presence of heparin. TGF-β binding efficiency were measured after overnight incubation of microparticles with TGF-β at 4 C. The release profile of TGF-β were measured by incubating the microparticles in PBS at room temperature under gentle mixing. ELISA kits were used to measure TGF-β concentrations.

FIG. 14 shows the formation of Tregs when a naïve CD4+ T cell is exposed to TGF-β and IL-2, leading to an iTreg cell (top left). The graphs show expression of CD25 and Foxp3 markers for hard and soft microparticles of different sizes (right). Cells with high level of CD25 and Foxp3 considered as Tregs as quantified here (bottom left).

FIG. 15 is a schematic depicting the inhibition of Treg formation (left) using TGF-β inhibitors galunisertib (LY2157299; molecular weight 369.42; IC50 56 nM) (top right) and SB505124 (molecular weight 335.4; IC50 129 nM) (bottom right).

FIG. 16 further describes TGF-β inhibitors galunisertib (LY2157299) and SB505124 (top). The graph (left) shows expression of Foxp3 as an indicator of Treg formation, while the graph (center) shows the quantification of flow cytometry graphs based on geometrical mean (Center) and the percentage of Foxp3+ T cells (right). Naïve T cells were co-cultured with microparticles and flow cytometry is being used after 4 days to evaluate the intracellular Foxp3 expression.

FIG. 17 depicts TGF-β inhibitors on microparticles. It shows the enhanced affinity of TGF-β inhibitors in the presence of β-cyclodextrin.

FIG. 18 further depicts TGF-β inhibitors. It shows molecular structure of galunisertib (LY2157299) and SB505124 as well as β-cyclodextrin (left top and bottom) and the molecular structure of the combined galunisertib and β-cyclodextrin (top right) and SB505124 and β-cyclodextrin (bottom right) following molecular dynamic simulation which showing their strong interaction while being inserted in β-cyclodextrin pocket.

FIG. 19 depicts activity of TGF-β inhibitors. The graph (left) shows release of galunisertib (LY2157299) from microparticles which modified chemically with β-cyclodextrin, and the molecular structures are the combined galunisertib and β-cyclodextrin, side view; center) and top view; right).

FIG. 20 depicts inhibition of Treg formation. The graphs show formation on Tregs in absence or presence of TGF-β inhibitor (left top and bottom) and also show the effects of TGF-β inhibitor being release by particles with mechanical properties and sizes (right) with respect to hard and soft microparticles of different sizes.

FIG. 21 further depicts inhibition of Treg formation. The graphs show the percentage of Tregs, 4 days after Naïve T cells were co-cultured with microparticles in the presence of TGF-β inhibitor-releasing microparticles to measure the change in formation of Tregs. Flow cytometry is being used after 4 days to evaluate the intracellular Foxp3 expression in activated (CD25+) CD4+ T cells as indicator of Tregs formation.

FIG. 22 depicts the dependence of CD8+ activation on mechanical properties of microparticles. B-cell membrane MPs were treated with SIINFEKL (SEQ ID NO: 1) (ovalbumin peptide) and cultured with naïve OT-I T cells prepared by negative selection from mouse spleens ex vivo. The MPs were loaded with IL-2 as well. After 4 days, cells were tested using flow cytometry via intracellular cytokine assay (ICCS) to assess secretion of pro-inflammatory cytokines (here IFN-γ).

FIG. 23 depicts the dependence of CD4+ and CD8+ expansion on mechanical properties as well as size of microparticles. B-cell membrane MPs were treated with ovalbumin peptides and cultured with naïve OT-I CD8+ or OT-II CD4+ T cells prepared by negative selection from mouse spleens ex vivo. The MPs were loaded with IL-2 as well. After 4 days, T cell expansion were tested using by cell counting.

FIG. 24 depicts the dependence of CD4+and CD8+activation on mechanical properties of microparticles. B-cell membrane MPs were treated with ovalbumin peptides and cultured with naïve OT-I CD8+ or OT-II CD4+ T cells prepared by negative selection from mouse spleens ex vivo. The MPs were loaded with IL-2 as well. After 4 days, change in cell numbers were assessed to quantify the expansion and also cells were tested using flow cytometry via ELISA cytokine assays to assess secretion of inflammatory cytokines (here IFN-γ and IL-2) (right).

FIG. 25 depicts the dependence of CD4+ and CD8+ expansion on mechanical properties of microparticles. B-cell membrane MPs were treated with ovalbumin peptides and cultured with naive OT-I CD8+ or OT-II CD4+ T cells prepared by negative selection from mouse spleens ex vivo. The MPs were loaded with IL-2 as well. After 7 and 14 days, change in number of CD4+ and CD8+ T cells were assessed by flow cytometry.

FIG. 26 demonstrates OT-I T cells were activated ex vivo with peptide-loaded cell membrane-coated microparticles for 4 days. 1×10⁵ B16F10-OVA cells subcutaneously injected into right flanks of C57BL/6J wild type (WT) mice (6-8 weeks old) mice. 5×10⁵ OT-I T cells were transferred intravenously on day 5 by retro-orbital injections (100 microliters [μL] per animal).

FIG. 27 shows the growth of B16F10-OVA melanoma tumors in mice intravenously administered T cells that were activated ex vivo with B-cell membrane nanoparticles treated with SIINFEKL.

FIG. 28 shows the masses of B16F10-OVA melanoma tumors in mice 22 days after tumor injection. Mice injected with T cells that were activated ex vivo with B-cell membrane nanoparticles treated with SIINFEKL.

FIG. 29 depict the percentage of CD8+ T cells (left) and granzyme B expressing CD8+ T cells (right) in tumor T cells in mice 22 days after tumor injection. Mice injected with T cells that were activated ex vivo with B-cell membrane nanoparticles treated with SIINFEKL.

FIG. 30 demonstrates OT-I T cells were activated ex vivo with B cell and B cell/B16F10-Ova membrane-coated microparticles for 4 days. 1×10⁵ B16 F10-OVA cells subcutaneously injected into right flanks of C57BL/6J wild type (WT) mice (6-8 weeks old) mice. 5×10⁵ OT-I T cells were transferred intravenously on day 5 by retro-orbital injections (100 microliters [μL] per animal).

FIG. 31 shows the masses of B16F10-OVA melanoma tumors in mice 22 days after tumor injection. Mice injected with T cells that were activated ex vivo B cell and B cell/B16F10-Ova membrane-coated microparticles for 4 days.

FIG. 32 depict the percentage of CD8+ T cells (left) and granzyme B expressing CD8+ T cells (right) in tumor T cells in mice 22 days after tumor injection. Mice injected with T cells that were activated ex vivo B cell and B cell/B16F10-Ova membrane-coated microparticles for 4 days.

FIG. 33 demonstrates B cell and B cell/B16F10-Ova membrane-coated microparticles for 4 days can activated endogenous T cells and fight tumor. 1×10⁵ B16 F10-OVA cells subcutaneously injected into right flanks of C57BL/6J wild type (WT) mice (6-8 weeks old) mice. 1×10⁶ B cell membrane-coated microparticles (with or without peptide) or B cell/B16-F10-Ova melanoma cell membrane-coated microparticles were injected subcutaneously injected close to tumor site on day 5. There is no administration of transgenic OT-I T cells here.

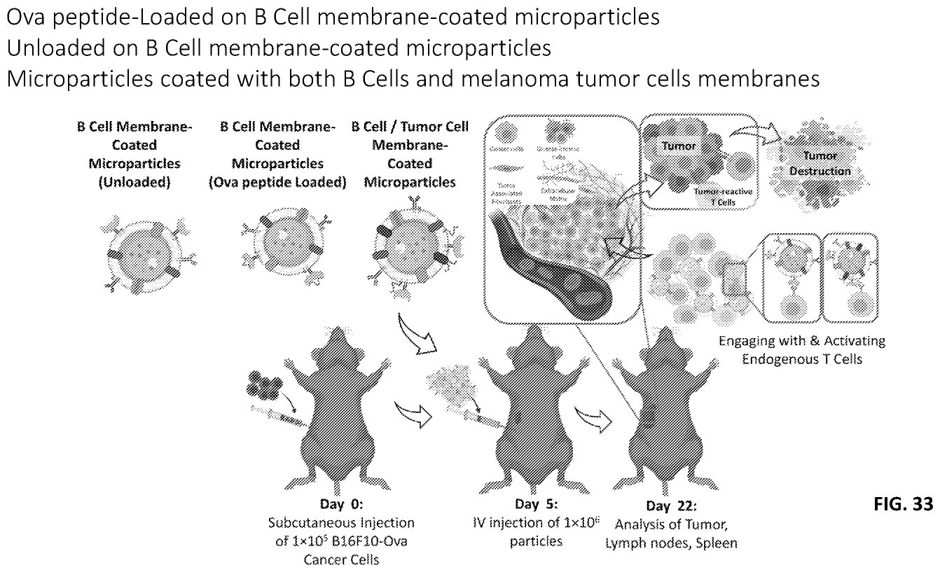

FIG. 34 shows the masses of B16F10-OVA melanoma tumors in mice 22 days after tumor injection. Mice injected with B cell membrane-coated microparticles (with or without peptide) or B cell/B16-F10-Ova melanoma cell membrane-coated microparticles were injected subcutaneously injected close to tumor site on day 5. There is no administration of transgenic OT-I T cells here.

FIG. 35 depict the percentage of CD8+ T cells (left) and granzyme B expressing CD8+ T cells (right) in tumor T cells in mice 22 days after tumor injection. Mice injected with B cell membrane-coated microparticles (with or without peptide) or B cell/B16-F10-Ova melanoma cell membrane-coated microparticles were injected subcutaneously injected close to tumor site on day 5. There is no administration of transgenic OT-I T cells here.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the following detailed description, numerous specific details are set forth in order to provide a thorough understanding of implantable scaffolds and microparticles, and the uses thereof. However, it will be understood by those skilled in the art that the production of these implantable scaffolds and microparticles and uses thereof may be practiced without these specific details. In other instances, well-known methods, procedures, and components have not been described in detail so as not to obscure their description.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized or customized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term “higher vertebrates” is used herein and includes avians (birds) and mammals. The compositions described herein can be used to treat any suitable mammal, including primates, such as monkeys and humans, horses, cows, cats, dogs, rabbits, sheep, goats, pigs, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

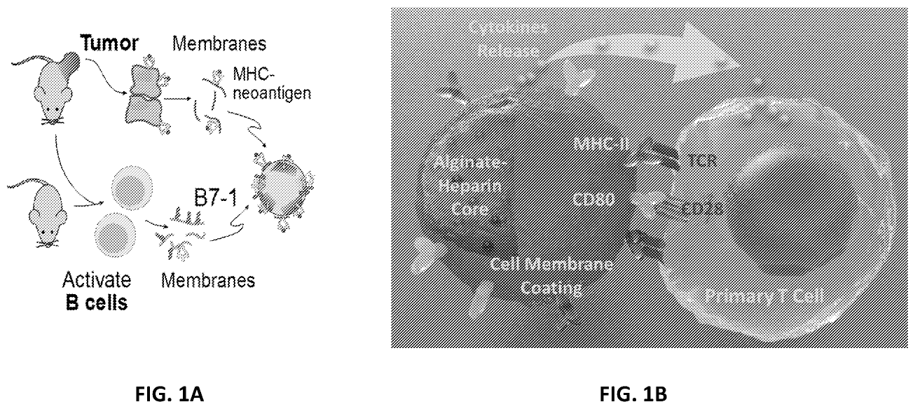

This invention is directed to novel microparticles comprising antigens of interest and antigen presenting cell (APC) components useful for stimulating the immune response, and in one embodiment, used for ex vivo amplification of cytotoxic T cells against a disease or medical condition. In many cases the antigen is unknown and laborious to determine, and in some instances, cells themselves may bear neoantigens expressed on MHC-I and II but lack costimulation or, worse, promote co-inhibitory pathways. As described in the examples herein, a new approach is demonstrated to generate artificial APCs (aAPCs) that promote T cell responses to tumors by combining the membranes of cells and the membranes of activated antigen presenting cells in a microparticle (FIG. IA). Ex vivo activation of T cells (e.g., as depicted in FIG. 1B) using such membrane-bearing particles will offer, in one embodiment, personalized, customized immunotherapy, as well as enabling specific targeting of localized conditions.

Activating large numbers of endogenous T cells to attack tumor neoantigens is conceptually straightforward and serves as the basis for a number of efforts in cancer and other vaccines. Identifying the neoantigen genetically or computationally is slow and has considerable hurdles to achieve real-time treatment. Despite earlier work on membrane-coated microparticles, tumor membranes themselves, however, lack costimulatory molecules and thus are completely ineffective at priming naïve T cells. In one embodiment, by grafting the membranes of activated APCs, such as but not limited to B cells, with membranes from tumor cells onto microparticles for activating T cells and expanding them ex vivo to massive numbers for adoptive immunotherapy. In another embodiment, such membrane-coated microparticles may be used to activate host immune cell in situ near the tumor site or in-line in a pump or leukapheresis device. The biodegradative nature of these particles can be tuned to provide an adequate therapeutic window before degradation.

Details of each component of the microparticles are described below.

### Microparticles

In some embodiments, described throughout herein, are “microparticles.” These microparticles may serve as a “platform” comprising an antigen and a costimulatory component derived from an antigen presenting cell. The antigen may comprise a tumor antigen; a cancer-specific antigen; a self antigen; an IgE receptor-specific antigen; a pathogen-specific antigen; an antigen specific to an organ, tissue, or cell of interest; an antigen specific to a transplanted organ, tissue, or cell of interest; a macrophage-specific antigen; an erythrocyte-specific antigen; a platelet-specific antigen; a platelet factor 5-specific antigen; a fibrinogen-specific antigen; a stem cell- or progenitor cell-specific antigen; a lymphocyte-specific antigen; a monocyte-specific antigen; an immune cell-specific antigen; or a combination thereof.

In non-limiting examples, the antigen may comprise a tumor antigen comprising a membrane isolated from a tumor cell or a tumor tissue; a cancer-specific antigen comprising a membrane isolated from a cancer cell or a cancer tissue; a self antigen comprising a membrane isolated from a cell, a tissue, or an organ targeted by an autoimmune disease or undergoing autoimmune attack; an IgE receptor-specific antigen comprising a membrane isolated from a mast cell or a basophil in response to an allergic reaction; hypersensitivity reaction, a pathogen-specific antigen comprising a membrane isolated from a cell of a pathogen or a viral envelope or a viral capsid; a macrophage-specific antigen or an immune cell (e.g., a B cell, a T cell, a natural killer [NK] cell, or a macrophage)-specific antigen comprising a membrane isolated from a macrophage or immune cell (e.g., a B cell, a T cell, a natural killer [NK] cell, or a macrophage, respectively, of interest) of interest; an antigen specific to an organ, tissue, or cell of interest comprising a membrane isolated from the organ, tissue, or cell of interest; an antigen specific to a transplanted organ, tissue, or cell of interest comprising a membrane isolated from the organ, tissue, or cell of interest; or an erythrocyte-specific antigen, a platelet-specific antigen, a platelet factor 5-specific antigen, a fibrinogen-specific antigen comprising a membrane isolated from an erythrocyte or a platelet (e.g., a thrombocyte); the antigen comprises a stem cell- or progenitor cell-specific antigen comprising a membrane isolated from a stem cell or a progenitor cell, respectively, of interest; a lymphocyte-specific antigen comprising a membrane isolated from a lymphocyte of interest; or a monocyte-specific antigen comprising a membrane isolated from a monocyte (e.g., a leukocyte) of interest.

The tumor cell or tumor tissue may be obtained from a surgically obtained tumor, tumor biopsy, or tumor sample. The cell, tissue, or organ of interest may be obtained from a surgically obtained cell, tissue, or organ of interest, a biopsy, or a sample. The antigen may also be obtained from a cell grown in vitro or a culture media thereof.

The costimulatory component from an antigen presenting cell may comprise a membrane isolated from an antigen presenting cell. The antigen presenting cell may be stimulated in vitro before the membrane is isolated.

An “antigen presenting cell” (APC) or “accessory cell” is a cell that displays antigen complexed with major histocompatibility complexes (MHCs) on its surface; this process is known as antigen presentation. T cells may recognize these complexes using their T cell receptors (TCRs). APCs process antigens and present them to T-cells. APCs include macrophages, B cells and dendritic cells, which present foreign antigens to helper T cells, while other cell types can present antigens originating inside the cell to cytotoxic T cells.

“Costimulatory components” are expressed on the membrane of the APC and the T cell. T cells require two signals to become fully activated. A first signal, which is antigen-specific, is provided through the T cell receptor (TCR) which interacts with peptide-MHC molecules on the membrane of antigen presenting cells (APC). A second signal, the co-stimulatory signal, is antigen nonspecific and is provided by the interaction between “co-stimulatory components” or “costimulatory molecules” expressed on the membrane of APC and the T cell. Examples of costimulatory components include, but are not limited to, the following: CD28 on T cells interacts with CD80 (B7.1) and CD86 (B7.2) on APC; ICOS (Inducible Costimulator) interacts with ICOS-L on APC; CD40L on T cells interacts with CD40 on APC; CTLA-4 on T cells interacts with CD80 and CD86 on APC; OX40 on T cells interacts with OX40L on APC; PD-1 on T cells interacts with PDL-L1 and PDL-L2 on APC.

In some embodiments, described throughout herein, are “microparticles.” These microparticles may serve as a “platform” comprising an antigen and a costimulatory component derived from an antigen presenting cell. Additionally, at least one compound that regulates induction of T regulatory cells (Tregs) may in certain embodiments be incorporated into the microparticles. Additionally, the microparticles may comprise a polymer, such as a biocompatible polymer, non-limiting examples of which include alginate, hyaluronic acid, or chitosan. Further, the microparticles may comprise heparin, In one non-limiting embodiment, the microparticles may comprises alginate and heparin. The polymer may be crosslinked. Still further, the microparticles may comprise paramagnetic nanoparticles, such as superparamagnetic iron oxide nanoparticles (SPIONs). Still further, the microparticles may also comprise at least one immunoregulatory compound. In some embodiments, the microparticle has a size comprising 1-1000 micrometers.

In some embodiments, microparticles may comprise a “coating” material. In some embodiments, these materials provide microparticles with enhanced biological characteristics, including interactions with cells and biomolecules. In some embodiments, microparticles are formed in the presence of a mix of alginate-heparin. In some embodiments, microparticles are formed in the presence of a mix of alginate. In some embodiments, an alginate may be sulfated.

A skilled artisan would appreciate that a description of a microparticle comprising an alginate or alginate-heparin coating may in certain embodiments, encompass a microparticle prepared in the presence of alginate or alginate and heparin, wherein these molecules and integral components of the microparticle synthesized.

In some embodiments, the microparticle further comprises at least one immunoregulatory compounds. In some embodiments, the at least one immunoregulatory compound comprises an immunostimulatory compound. In other embodiments, the at least one immunoregulatory compound comprises an immunosuppression compound. In some embodiments, the at least one immunoregulatory compound comprises a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid. In some embodiments, the cytokine comprises an interleukin (IL). In some embodiments, the cytokine comprises interleukin-2 (IL-2), interleukin-12 (IL-12), or interleukin-15 (IL-15). In some embodiments, the chemokine comprises stromal cell-derived factor 1a (SDF-1a). In some embodiments, the growth factor comprises transforming growth factor-beta (TGF-β), vascular endothelial growth factor (VEGF), or bone morphogenetic protein-2 (BMP-2). In some embodiments, the microparticle comprises IL-2 and TGF-β.

In some embodiments, the microparticle further comprises at least one compound that regulates induction of regulatory T cells (Tregs). In some embodiments, the at least one compound that regulates induction of Tregs comprises a compound that suppresses induction of Tregs. In some embodiments, the compound that suppresses induction of Tregs comprises a TGF-β inhibitor. In some embodiments, the TGF-β inhibitor is a TGF-β receptor inhibitor. In some embodiments, the TGF-β inhibitor is galinusertib (LY2157299) or SB505124. In other embodiments, the at least one compound that regulates induction of Tregs comprises a compound that induces Tregs. In some embodiments, the compound that induces Tregs is a TGF-β or an activator thereof.

In some embodiments, the microparticle further comprises a small molecule. In some embodiments, the microparticle further comprises a hydrophobic therapeutic agent.

The microparticles are generated by microfluidic droplet generation and may be made from a biocompatible polymer such as but not limited to alginate, hyaluronic acid and chitosan, or any combination thereof, and may be cross-linked. Heparin may be included. In one example, using alginate-heparin as the main component, the alginate is crosslinked using a 4-arm PEG hydrazide cross linker. In one embodiment, superparamagnetic nanoparticles may be embedded in the microparticles. To adjust the microparticle size, the flow ratio in the generator can be adjusted for different droplet diameters, which translate to particle sizes (and dispersities).

In one embodiment, the microparticles are less than about 5 μm. In one embodiment, the microparticles are 0.15 to 5 μm. In one embodiment the microparticles are about 4 μm. In one embodiment, the microparticles are about 1.33 μm. In one embodiment the microparticles are about 0.57 μm.

By varying the amount of crosslinker, the particles can be made mechanically soft or stiff. In one embodiment, the microparticles have a Young's modulus of less than about 25 kPa. In one embodiment, the microparticles have a Young's modulus of less than 5 kPa. In one embodiment the microparticles have a Young's modulus of about 20 kPa. In one embodiment, the microparticles have a Young's modulus of about 3 kPa.

Other components may be included in the microparticles such as one or more cytokines, chemokines, growth factors and antibodies. Non-limiting examples include cytokines such as IL-2, IL-12, and IL-15; chemokines such as SDF-1a; growth factors such as TGF-β, VEGF and BMP-2. Additional components useful for immunoregulatory are IL-2 and TGF-β. IL-2 may be included to the microparticles for uptake and later, passive release, to promote T-cell activation. By including heparin in the microparticle formulation, more IL-2 can be retained by the particles and released more slowly.

Paramagnetic nanoparticles may be included in the microparticles, e.g., for purification or for ease of separation from leukocytes following exposure of the leukocytes to the microparticles. In some embodiments, the paramagnetic nanoparticles comprise superparamagnetic iron oxide nanoparticles (SPIONs). In some embodiments, a SPION comprises a particle having a size about 50-200 mm. This addition may in certain embodiments enhance purification of microparticles using methods well known in the art.

### Membrane Components

Membrane component from tumor cells. Tumor cells obtained from biopsy or tumor resection, or grown in vitro, may be used to generate membrane fragments to add to the microparticles. Cells may be lysed with a lysis buffer and membranes collected by centrifugation. Membranes are then incubation with the microparticles. Uptake of membranes can be monitored such as described in the examples.

Membrane component from antigen presenting cells (APCs). B cells can be activated in vitro, for example with LPS, lysed with a lysis buffer and membranes collected by centrifugation. Membranes are then incubation with the microparticles. Uptake of APC membrane components on the microparticles can be monitored such as described in the examples. A membrane may be isolated from a macrophage, a B cell, a T cell, or a natural killer (NK) cell of interest.

Membrane components from self-antigens. As will be described in another embodiment, microparticles for the purpose of eliciting T regulatory cells may comprise self-antigen membrane components. Tissues undergoing autoimmune attack may be biopsied and membranes prepared therefrom, for incorporation into the microparticles. In this embodiment, microparticles may also be loaded with TGF-β and IL-2, to induce Tregs.

Other membrane components. In other non-limiting embodiments, a membrane may be isolated from a mast cell or a basophil following response to an allergic reaction or hypersensitivity reaction; a membrane may be isolated from a cell of a pathogen or a viral envelope or a viral capsid; a membrane may be isolated from a macrophage, a B cell, a T cell, or a natural killer (NK) cell of interest; a membrane may be isolated from a stem cell or a progenitor cell of interest; a membrane may be isolated from a transplanted organ, tissue, or cell of interest; a membrane may be isolated from a specific organ, tissue, or cell of interest; or a membrane isolated from an erythrocyte (red blood cell), a monocyte (e.g., leukocyte) or a platelet (e.g., a thrombocyte) of interest.

### Other Components

As will be described below, other embodiments of the invention provide additional components of the microparticles for certain purposes.

### TGF-β Inhibitors

A TGF-β inhibitor (TGF-βi) such as a TGF-β receptor inhibitor may be used in the microparticles to, in one embodiment, discourage the formation of regulatory T cells (Tregs). Non-limiting examples include galinusertib (LY157299) or SB505124. The compound is incorporated into the microparticle. In one embodiment, the TGF-βi suppresses the formation of induced Tregs and thus enhances the tumoricidal activity of T cells attracted to, activated, or delivered by the microparticles described herein. In one embodiment the TGF-β inhibitor is slowly released from the microparticle.

### TGF-β

In another embodiment, microparticles may comprise TGF-β for the purpose of inducing formation of Tregs. IL-2 may also be included. Additionally, an activator of TGF-β.

### Additional Components

Microparticles of the invention may also include other components, such as, but not limited to, cytokines, growth factors, peptides, and nucleic acids such as but not limited to DNA, RNA.

### T Cells and Regulatory Compounds

In some embodiments, immune cells, for example T cell, are generated and expanded by the presence of cytokines in vivo. In some embodiments, cytokines that affect generation and maintenance to T-helper cells in vivo comprise IL-2, IL-12, and IL-15. In some embodiments, T regulatory (Treg) cells are generated from naïve T cells by cytokine induction in vivo. In some embodiments, TGF-β and/or IL-2 play a role in differentiating naïve T cell to become Treg cells.

“Cytokines” are a category of small proteins (˜5-20 kDa) critical to cell signaling. Cytokines are peptides and usually are unable to cross the lipid bilayer of cells to enter the cytoplasm. Among other functions, cytokines may be involved in autocrine, paracrine and endocrine signaling as immunomodulating agents. Cytokines may be pro-inflammatory or anti-inflammatory. Cytokines include, but are not limited to, chemokines (cytokines with chemotactic activities), interferons, interleukins (ILs; cytokines made by one leukocyte and acting on one or more other leukocytes), lymphokines (produced by lymphocytes), monokines (produced by monocytes), and tumor necrosis factors. Cells producing cytokines include, but are not limited to, immune cells (e.g., macrophages, B lymphocytes, T lymphocytes and mast cells), as well as endothelial cells, fibroblasts, and various stromal cells. A particular cytokine may be produced by more than one cell type.

A skilled artisan would appreciate that the term “cytokine” may encompass cytokines beneficial to enhancing an immune response targeted against a cancer or a pre-cancerous or non-cancerous tumor or lesion. A skilled artisan would also appreciate that the term “cytokine” may encompass cytokines beneficial to enhancing an immune response against a disease or inflammation (e.g., resulting from surgery, an injury, or damage from an autoimmune response) or that the term “cytokine” may encompass cytokines beneficial to reducing an abnormal autoimmune response.

In some embodiments, a cytokine encoded by the nucleic acid expands and maintains T-helper cells (helper T cells). In some embodiments, a cytokine encoded by the nucleic acid expands T-helper cells. In some embodiments, a cytokine encoded by the nucleic acid maintains T-helper cells. In some embodiments, a cytokine encoded by the nucleic acid expands cytotoxic T cells (CTLs). In some embodiments, a cytokine encoded by the nucleic acid activates cytotoxic T cells. In some embodiments, a cytokine encoded by the nucleic acid expands and activates cytotoxic T cells. In some embodiments, a cytokine encoded by the nucleic acid increases proliferation of a T-helper cell population. In some embodiments, a cytokine encoded by the nucleic acid increases proliferation of a cytotoxic T cell population.

In some embodiments, the encoded cytokine comprises an interleukin (IL). A skilled artisan would appreciate that interleukins comprise a large family of molecules, including, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

In some embodiments, the encoded interleukin comprises an IL-2, IL-4, IL-6, IL-7, IL-10, IL-12, or an IL-15, or any combination thereof. In some embodiments, the encoded cytokine comprises an IL-2. In some embodiments, the encoded cytokine comprises an IL-4. In some embodiments, the encoded cytokine comprises an IL-6. In some embodiments, the encoded cytokine comprises an IL-7. In some embodiments, the encoded cytokine comprises an IL-10. In some embodiments, the encoded cytokine comprises an IL-12. In some embodiments, the encoded cytokine comprises an IL-15.

In some embodiments, the plasmid encodes any additional cytokine or polypeptide or peptide.

In some embodiments, the IL-2 cytokine comprises an IL-2 superkine (super IL-2 cytokine, Super2). IL-2 is a 133 amino acid glycoprotein with one intramolecular disulfide bond and variable glycosylation.

“IL-2 superkine” or “Super2” (Fc) is an artificial variant of IL-2 containing mutations at positions L80F/R81D/L85V/I86V/I92F. These mutations are located in the molecule's core that acts to stabilize the structure and to give it a receptor-binding conformation mimicking native IL-2 bound to CD25. These mutations effectively eliminate the functional requirement of IL-2 for CD25 expression and elicit proliferation of T cells. Compared to IL-2, the IL-2 superkine induces superior expansion of cytotoxic T cells, leading to improved antitumor responses in vivo, and elicits proportionally less toxicity by lowering the expansion of T regulatory cells and reducing pulmonary edema. Examples of IL-2 superkine (Super2) deoxyribonucleic acid (DNA) and protein sequences can be found, e.g., in Table 1.

TABLE 1
IL-2 superkine (Super2) Sequence.

| Type of | Sequence |
|---|---|
| Sequence | (SEQ ID NO) |
| Super2 | GGAGCCATGGGAGAATTCGCACCTACTTCAAGTT |
| nucleotide | CTACAAAGAAAACACAGCTACAACTGGAGCATT |
| sequence | TACTTCTGGATTTACAGATGATTTTGAATGGAAT |
| | TAATAATTACAAGAATCCCAAACTCACCAGGAT |
| | GCTCACATTTAAGTTTTACATGCCCAAGAAGGCC |
| | ACAGAACTGAAACATCTTCAGTGTCTAGAAGAA |
| | GAACTCAAACCTCTGGAGGAAGTGCTAAATTTA |
| | GCTCAGAGCAAAAACTTTCACTTCGATCCCAGGG |
| | ACGTCGTCAGCAATATCAACGTATTCGTCCTGGA |
| | ACTAAAGGGATCTGAAACAACATTCATGTGTGA |
| | ATATGCTGATGAGACAGCAACCATTGTAGAATTT |
| | CTGAACAGATGGATTACCTTTTGTCAAAGCATCA |
| | TCTCAACACTAACTCAT (SEQ ID NO: 2) |
| Super2 | MGEFAPTSSSTKKTQLQLEHLLLDLQMILNGINNY |
| protein | KNPKLTRMLTFKFYMPKKATELKHLQCLEEELKPL |
| sequence | EEVLNLAQSKNFHFDPRDVVSNINVFVLELKGSETT |
| | FMCEYADETATIVEFLNRWITFCQSIISTLTH (SEQ |
| | ID NO: 3) |

A “T cell” is characterized and distinguished by the T cell receptor (TCR) on the surface. A T cell is a type of lymphocyte that arises from a precursor cell in the bone marrow before migrating to the thymus, where it differentiates into one of several kinds of T cells. Differentiation continues after a T cell has left the thymus. A “cytotoxic T cell” (CTL) is a CD8+ T cell able to kill, e.g., virus-infected cells or cancer cells. A “T helper cell” is a CD4+ T cell that interacts directly with other immune cells (e.g., regulatory B cells) and indirectly with other cells to recognize foreign cells to be killed. “Regulatory T cells” (T regulatory cells; Treg), also known as “suppressor T cells,” enable tolerance and prevent immune cells from inappropriately mounting an immune response against “self,” but may be co-opted by cancer or other cells. In autoimmune disease, “self-reactive T cells” mount an immune response against “self” that damages healthy, normal cells.

One skilled in the art appreciates the many mechanisms of T cell immunostimulation and/or immunosuppression. Likewise, one skilled in the art appreciates the many mechanisms of Treg induction and/or suppression of Treg induction.

T cell immunostimulatory compounds include, but are not limited to, T cell activators, T cell attractants, or T cell adhesion compounds. T cell immunostimulatory compounds include, but are not limited to, cytokines, chemokine ligands, and anti-CD antibodies or fragments thereof. Non-limiting examples include interleukins (e.g., IL-2, IL-12, or IL-15), chemokine ligands (e.g., CCL ligands, including CCL21), and anti-CD antibodies (e.g., anti-CD3 or anti-CD28) or fragments thereof, or any combination(s) thereof.

T cell immunosuppression compounds include, but are not limited to cytokines, chemokines, antibodies, or enzymes.

Compounds that suppression induction of Tregs include, but are not limited to, inhibitors of transforming growth factor-beta (TGF-β), such as an inhibitor of the TGF-β receptor. Non-limiting examples of TGF-β receptor inhibitors include galinusertib (LY2157299), SB505124, small molecule inhibitors, antibodies, chemokines, apoptosis signals (e.g., cytotoxic T-lymphocyte-associated protein 4/programmed cell death protein 1 (CTLA-4/PD-1); Granzyme; tumor necrosis factor (TNF)-related apoptosis-inducing ligand (TRAIL); Fas/Fas-L, Galectin-9/transmembrane immunoglobulin and mucin domain 3 (TIM-3)). Compounds that induce Tregs include TGF-β and activators thereof (e.g., SB 431542, A 83-01, RepSox, LY 364947, D 4476, SB 525334, GW 788388, SD 208, R 268712, IN 1130, SM 16, A 77-01, AZ 12799734).

As used herein, a “targeting agent,” or “affinity reagent,” is a molecule that binds to an antigen or receptor or other molecule. In some embodiments, a “targeting agent” is a molecule that specifically binds to an antigen or receptor or other molecule. In certain embodiments, some or all of a targeting agent is composed of amino acids (including natural, non-natural, and modified amino acids), nucleic acids, or saccharides. In certain embodiments, a “targeting agent” is a small molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cγ2), and C-gamma-3 (Cγ3). In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cyγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies, and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)).

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri(a)body, a di- or tri-tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BV” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983); Morley, J. S., “Trends Pharm Sci.” (1980) pp. 463-468 ; Hudson et al., Int. J. Pept. Prot. Res. (1979) 14, 177-185; Spatola et al., Life Sci. (1986) 38, 1243-1249; Hann, M. M., J. Chem. Soc. Perkin Trans. I (1982) 307-314; Almquist et al., J. Med. Chem. (1980) 23, 1392-1398; Jennings-White et al., Tetrahedron Lett. (1982) 23, 2533; Szelke et al., European Application EP 45665; Chemical Abstracts 97, 39405 (1982); Holladay, et al., Tetrahedron Lett. (1983) 24, 4401-4404; and Hruby, V. J., Life Sci. (1982) 31, 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10'5 M or less than about 1×10−6 M or 1×10−7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In addition to antibody sequences, an antibody according to the present invention may comprise other amino acids, e.g., forming a peptide or polypeptide, such as a folded domain, or to impart to the molecule another functional characteristic in addition to ability to bind antigen. For example, antibodies of the invention may carry a detectable label, such as fluorescent or radioactive label, or may be conjugated to a toxin (such as a holotoxin or a hemitoxin) or an enzyme, such as beta-galactosidase or alkaline phosphatase (e.g., via a peptidyl bond or linker).

In one embodiment, an antibody of the invention comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises a C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

In one embodiment, a variety of radioactive isotopes are available for the production of radioconjugate antibodies and other proteins and can be of use in the methods and compositions provided herein. Examples include, but are not limited to, At211, Cu64, I131, I125, Y90, Re186, Re188, Sm153, Bi212, P32, Zr89 and radioactive isotopes of Lu. In a further embodiment, the amino acid sequences of the invention may be homologues, variants, isoforms, or fragments of the sequences presented. The term “homolog” as used herein refers to a polypeptide having a sequence homology of a certain amount, namely of at least 70%, e.g. at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified. The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide of the invention which has less amino acids than the full-length amino acid sequence of a polypeptide of the invention. The fragment may or may not possess a functional activity of such polypeptides.

In an alternate embodiment, enzymatically active toxin or fragments thereof that can be used in the compositions and methods provided herein include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

A chemotherapeutic or other cytotoxic agent may be conjugated to the protein, according to the methods provided herein, as an active drug or as a prodrug. The term “prodrug” refers to a precursor or derivative form of a pharmaceutically active substance that is less cytotoxic to tumor cells compared to the parent drug and is capable of being enzymatically activated or converted into the more active parent form. (See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.) The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

Non-limiting examples of antibodies, antibody fragments and antigen-binding proteins include single-chain antibodies such as scFvs. A non-limiting example, a scFv that blocks PD-1 for the treatment of cancer or tumor, including in association with CAR-T therapy, wherein activation of scFv production can be directed at a particular site in the body, in one embodiment, at or near a tumor. Another non-limiting example includes brolucizumab, which targets VEGF-A and is used to treat wet age-related macular degeneration.

In another example, the therapeutic protein is an immune checkpoint inhibitor, such as an antibody fragment, or antigen-binding protein, that inhibits a checkpoint molecule, such as, but not limited to, PD-1, PD-L1, CTLA-4, CTLA-4 receptor, PD1-L2, 4-1BB, OX40, LAG-3, and TIM-3. In one embodiment, a scFv that inhibits a checkpoint protein.

### Cell Adhesion/Attraction Components

Any one or more cell adhesion and/or cell attraction and/or immunostimulatory and/or immunosuppression compounds or components may be included in the microparticles or as part of the treatments described herein. In one embodiment, such components attract or activate T cells. Non-limiting examples include CCL21, anti-CD3 antibodies, anti-CD28 antibodies, or any combination thereof. In one embodiment, a combination of anti-CD3 and an anti-CD28 antibodies are used. Any one or more immunostimulatory components may be included. In some embodiments, components such as but not limited to IL-2, IL-4, IL-6, IL-7, IL-10, IL-12 and IL-15 are used, singly or in any combination. In other embodiments, such compounds or components or others (e.g., anti-CD3 or anti-CD28 antibodies) suppress T cell attraction or T cell activation. Additional embodiments are described elsewhere herein.

### Treg Regulators

Any one of various methods of regulating Treg induction and/or suppression of Treg induction may be used.

A TGF-β inhibitor (TGF-βi) such as a TGF-β receptor inhibitor may be used. Non-limiting examples include galinusertib (LY2157299) or SB505124. The compound is incorporated into the scaffold. In one embodiment, the TGF-βi suppresses the formation of induced Tregs and thus enhances the tumoricidal activity of T cells attracted to, activated, or delivered by the scaffolds described herein. In one embodiment the TGF-β inhibitor or inducer is slowly released from the microparticles. Alternatively, compounds that induce Tregs may be used. Non-limiting examples include TGF-β and activators thereof (e.g., IL-2, IL-4, IL-6, IL-7, IL-10, IL-12, IL-15). Additional embodiments are described elsewhere herein.

### Methods of Making Microparticles and Nanoparticles

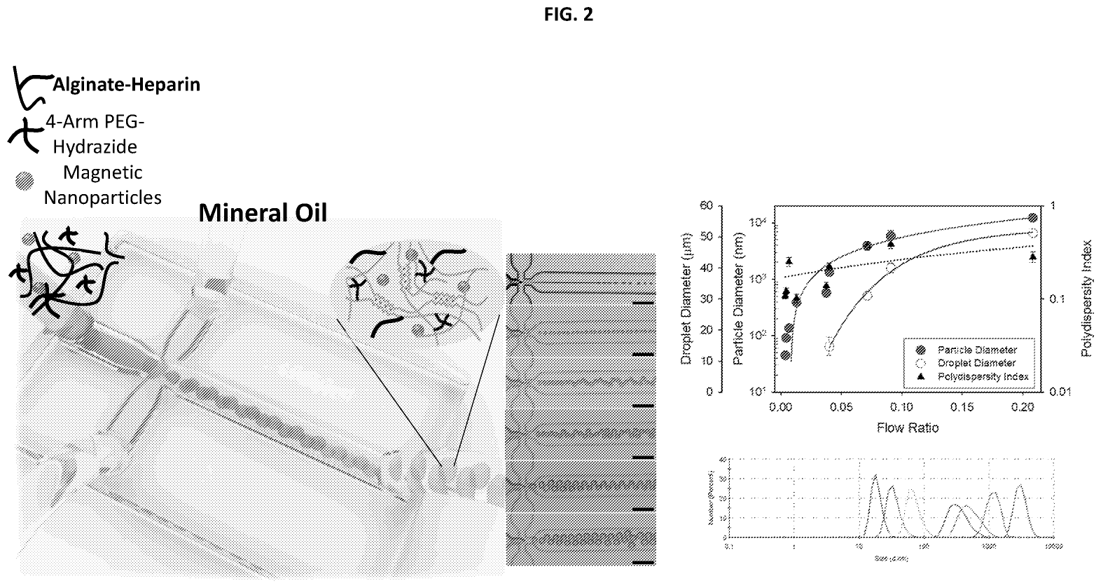

As described above, in one embodiment, a microfluidic droplet generator can be used to prepare the microparticles of the invention (see, e.g., FIG. 2). Other methods of generating particles are embodied herein.

This procedure can be used for both microparticles and nanoparticles with an overall size range of about 20 nm to about 300 microns (μm).

In some embodiments, the microparticles further comprise nanoparticles, such as paramagnetic nanoparticles (FIG. 2).

In some embodiments, the method comprises making and using nanoparticles with a microfluidic droplet generator to prepare immunoactive nanoparticles in lieu of microparticles.

In one non-limiting example, monodisperse mesoporous silica microparticles (5 to 20 μm [micrometers/microns]) were formed using a microfluidic jet spray-drying route, using cetyltrimethylammonium bromide (CTAB) and/or Pluronic F127 as templating agents, and tetraethylorthosilicate (TEOS) for silica as reported (see, e.g., Waldron, K. et al. Formation of monodisperse mesoporous silica microparticles via spray-drying. J. Colloid Interface Sci. (2014). doi:10.1016/j.jcis.2013.12.027; Liu, W., Chen, X. D. & Selomulya, C. On the spray drying of uniform functional microparticles. Particuology (2015). doi:10.1016/j.partic.2015.04.001). Carbodiimide chemistry (1-ethyl-3-(3 -dimethylaminopropyl)carbodiimide hydrochloride [EDC]/N-hydroxysuccinimide [NHS]; EDC/NHS) was utilized to modify silica conjugates with heparin after treating the silica with (3-Aminopropyl)triethoxysilane (APTES) to provide primary amine groups. Briefly, mesoporous silica microparticles (800 mg) was suspended in dehydrated Methanol (50 ml). Then, APTES (3 ml) was added and the suspension was stirred at room temperature overnight, and the final product was centrifuged (1500 rpm, 3 min) and washed with methanol five times, followed by drying under high vacuum. For the surface functionalization of the aminated-silica particles with heparin, heparin sodium salt (216 mg) was dissolved in deionized water (8 ml) and activated via successive addition of EDC (63 mg) and N-hydroxysulfosuccinimide (sulfo-NHS; 71.4 mg). After stirring for 5 min, the ethanolic solution of amino-functionalized silica (20 mg in 1.12 ml) was added to the reaction mixture and stirred for 12 hours (h) at room temperature. Afterwards the particles were separated by centrifugation and washed several times with deionized water and ethanol to remove unreacted reagents.

For the preparation of, e.g., antibody-conjugated microparticles, anti-CD3 (clone 2C11; BIO-X-CELL™) and anti-CD28 (clone 37.51; BIO-X-CELL™) were covalently conjugated to the surface of particles using carbodiimide chemistry. After activation of antibodies' carboxylic groups for 10 min with EDC/NHS, microparticles were added and incubated under gentle stirring at 4° C. (degrees Celsius) overnight. The protein-functionalized microparticles (artificial antigen presenting cells, aAPCs) were then separated from the solution and washed several times. Unreacted functional groups were quenched by washing samples in Tris buffer (100 mM, pH 8) for 30 min. A 10-fold dilution of the conjugation density that is used in a conventional plate-bound stimulation method for T cell activation was selected as the final conjugation density for beads. Micro-bicinconinic acid (MICRO-BCA™) assay was used to quantify total amount of surface conjugated antibodies according to the manufacturer's protocol.

To prepare IL-2 loaded aAPCs, microparticles were incubated with cytokine in PBS buffer containing bovine serum albumin (BSA; 0.1%w/v) and were gently shaken overnight at 4° C. The microparticles were then centrifuged and washed several times to remove unabsorbed cytokines. The concentration of IL-2 in the removed supernatant was measured using enzyme-linked immunosorbant assay (ELISA) to estimate the binding capacity of microparticles.

### Methods of Using Microparticles

In one embodiment, the microparticles of the invention comprising components to stimulate a cytotoxic T cell response to tumor antigens may be used ex vivo by exposing leukocytes isolated from a patient's whole blood (or collected by pheresis) to microparticles in order to induce and amplify the population of tumor specific T cells in the population. After induction, the cells may be separated from the microparticles (by use of the paramagnetic nanoparticles incorporated into the microparticles) and the cells infused into the patient. T cell therapy may be accompanied by other treatments to enhance the T cells, or attack the tumor by other mechanisms. In-line or leukapheresis devices incorporating the microparticles of the invention may also be provided.

“Apheresis” or “pheresis” comprises an ex vivo blood purification procedure during which a patient's blood is subjected to a separation apparatus or technique ex vivo to separate out a given constituent prior to the reinfusion of the blood back into the patient (or a different patient). “Leukapheresis” comprises apheretic separation of leukocytes from the blood.

In one embodiment microparticles can be targeted to and be bound to T cells during a leukapheresis or other blood cell purification procedure and infused into the patient. Such targeting binding of microparticles to leukocytes or other cell types after administration to the body or during a leukapheresis procedure or other ex vivo procedure provides the therapeutic protein in association with a cell type to effect its desired function.

Any of various diseases or medical conditions may be treated by the methods described herein. In one non-limiting embodiment, for treatment of solid tumors, inclusion of one or more inhibitors of TGF-β is desirable to avoid or suppress the induction of regulatory T cells. In another non-limiting embodiment, for treatment of autoimmune diseases, inclusion of TGF-β or of one or more activators thereof is desirable to induce regulatory T cells. The methods described herein are of particular use in situations involving treatments of inoperable or inaccessible targets of interest (e.g., an inoperable tumor) or in situations in which it is particularly desirable to target a specific cell population located in multiple, discreet areas of the body.

In some embodiments, “treating” comprises therapeutic treatment including prophylactic or preventive measures, wherein the object is to prevent or lessen the targeted pathologic condition or disorder, for example to treat or prevent cancer. Thus, in some embodiments, treating may include directly affecting or curing, suppressing, inhibiting, preventing, reducing the severity of, delaying the onset of, reducing symptoms associated with cancer or a combination thereof. Thus, in other embodiments, treating may include directly affecting or curing, suppressing, inhibiting, preventing, reducing the severity of, delaying the onset of, reducing symptoms associated with a non-cancerous tumor or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “cancer” is one of a group of diseases characterized by uncontrollable growth and having the ability to invade normal tissues and to metastasize to other parts of the body. Cancers have many causes, including, but not limited to, diet, alcohol consumption, tobacco use, environmental toxins, heredity, and viral infections. In most instances, multiple genetic changes are required for the development of a cancer cell. Progression from normal to cancerous cells involves a number of steps to produce typical characteristics of cancer including, e.g., cell growth and division in the absence of normal signals and/or continuous growth and division due to failure to respond to inhibitors thereof; loss of programmed cell death (apoptosis); unlimited numbers of cell divisions (in contrast to a finite number of divisions in normal cells); aberrant promotion of angiogenesis; and invasion of tissue and metastasis.

A “pre-cancerous” condition, lesion, or tumor is a condition, lesion, or tumor comprising abnormal cells associated with a risk of developing cancer. Non-limiting examples of pre-cancerous lesions include colon polyps (which can progress into colon cancer), cervical dysplasia (which can progress into cervical cancer), and monoclonal monopathy (which can progress into multiple myeloma). Premalignant lesions comprise morphologically atypical tissue which appears abnormal when viewed under the microscope, and which are more likely to progress to cancer than normal tissue.

A “non-cancerous tumor” or “benign tumor” is one in which the cells demonstrate normal growth, but are produced, e.g., more rapidly, giving rise to an “aberrant lump” or “compact mass,” which is typically self-contained and does not invade tissues or metastasize to other parts of the body. Nevertheless, a non-cancerous tumor can have devastating effects based upon its location (e.g., a non-cancerous abdominal tumor that prevents pregnancy or causes a ureter, urethral, or bowel blockage, or a benign brain tumor that is inaccessible to normal surgery and yet damages the brain due to unrelieved pressure as it grows).

In some embodiments, “treating” comprises therapeutic treatment including prophylactic or preventive measures, wherein the object is to prevent or lessen the targeted pathologic condition or disorder, for example to treat or prevent an autoimmune disease, an allergic reaction, a hypersensitivity reaction, a localized infection or an infectious disease, an injury or other damage, a transplant or other surgical site, or a symptom thereof, or a combination thereof. Thus, in some embodiments, treating may include directly affecting or curing, suppressing, inhibiting, preventing, reducing the severity of, delaying the onset of, reducing symptoms associated with an autoimmune disease, an allergic reaction, a hypersensitivity reaction, a localized infection or an infectious disease, an injury or other damage, a transplant or other surgical site, or a symptom thereof, or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “focus of interest” a “localized environment,” or a “localized site” comprises a site in which the disease, reaction, infection, injury, or other medical condition is specific to one part or area of the body; in which a symptom or condition of the medical condition is specific to one part or area of the body; or in which treatment is desired for one part or area of the body (even if the disease, reaction, infection, injury, or other medical condition affects other parts or areas of the body or the body as a whole).

As used herein, the terms “composition” and “pharmaceutical composition” may in some embodiments, be used interchangeably having all the same qualities and meanings. In some embodiments, disclosed herein is a pharmaceutical composition for the treatment of a cancer or tumor as described herein. In some embodiments, disclosed herein is a pharmaceutical composition for the treatment of cancer or tumor. In some embodiments, disclosed herein is a pharmaceutical composition for the use in methods locally regulating an immune response. In some embodiments, disclosed herein are pharmaceutical compositions for the treatment of an autoimmune disease, an allergic reaction, a hypersensitivity reaction, a localized site of an infection or infectious disease, a localized site of an injury or other damage, a transplant or other surgical site, a blood clot causing or at risk for causing a myocardial infarction, an ischemic stroke, or a pulmonary embolism or a symptom thereof, or a combination thereof.

### Preparation of an Activated Cytotoxic T Cell Population

Provided herein are methods for the preparation of an activated T cell population specific for a tumor antigen. Here, the microparticles described above are prepared. The antigen is obtained from a sample from the patient or other subject, as described above, and leukocytes are obtained from whole blood or pheresis, e.g., of blood from a patient or other subject. The leukocytes are exposed in vitro or ex vivo to the microparticles, which are then separated from the leukocytes, e.g., by magnetic separation or by other methods of separation known in the art. The treated leukocytes are then reinfused into the patient or other subject.

### Immune Response Stimulation or Suppression

In one embodiment, the microparticles can be used to stimulate the immune response. In another embodiment, the microparticles can be used to deliver signals to suppress the immune response. In a non-limiting example, by using immunostimulatory compounds, the method will provide signals to stimulate the immune response, e.g., as treatment for a tumor, cancer, infection, or infectious disease. In a non-limiting example, by using immunosuppression compounds, rather than immunostimulatory compounds, the method will provide signals to suppress the immune response, e.g., as treatment for an autoimmune disease or after organ transplantation.

In some embodiments, the disease or medical condition comprises a tumor or a cancer, and the focus of interest comprising the tumor or the cancer; the disease or medical condition comprises an autoimmune disease, and the focus of interest comprising an autoimmune-targeted or symptomatic focus of said autoimmune disease; the disease or medical condition comprises an allergic reaction or hypersensitivity reaction, and the focus of interest comprising a reactive focus of said allergic reaction or hypersensitivity reaction; the disease or medical condition comprises a localized infection or an infectious disease, and the focus of interest comprising a focus of infection or symptoms; the disease or medical condition comprises an injury or a site of chronic damage, and the focus of interest comprising the injury or the site of chronic damage; the disease or medical condition comprises a surgical site, and the focus of interest comprising the surgical site; the disease or medical condition comprises a transplanted organ, tissue, or cell, and the focus of interest comprising a transplant site; or the disease or medical condition comprises a blood clot causing or at risk for causing a myocardial infarction, an ischemic stroke, or a pulmonary embolism, and the porous scaffold is provided at or adjacent to a focus of interest comprising the site of the blood clot.

In a non-limiting example, by including a TGF-β inhibitor (e.g., TGFβi), the method will provide signals to suppress the formation of regulatory T cells (Tregs). In a non-limiting example, by using TGF-β or an activator thereof, instead of a TGF-β inhibitor, in the formulation, the scaffolds will provide signals to promote formation of regulatory T cells (Tregs).

In some embodiments, the autoimmune disease includes, for example, but is not limited to, rheumatoid arthritis, juvenile dermatomyositis, psoriasis, psoriatic arthritis, sarcoidosis, lupus, Crohn's disease, eczema, vasculitis, ulcerative colitis, multiple sclerosis, or type 1 diabetes, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital hear block, Coxsackie myocarditis, CREST syndrome, Crohn' s disease, dermatitis herpetiformis, dermatomyositis, Devic' s disease (neuromyelitis optica), discoid lupus, Dressler' s syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis) giant cell myocarditis, glomerulonephritis, Goodpasture's syndrome, granulomatosis with polyangiitis, Grave's disease, Guillain-Barre syndrome, Hashimoto's thyroiditis, hemolytic anemia, Henoch-Schonlein purpura (HSP), Herpes gestationis or pemphigoid gestationis (PG), Hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease, lupus, Lyme disease chronic, Menier's disease, microscopic polyangiitis (MPA), mixed connective tissue disease (MCTD), Mooren's ulcer, Mucha-Habermann disease, multifocoal motor neuropathy (MMN, MMNCB), multiple sclerosis, myasthenia gravis, myositis, narcolepsy, neonatallupus, neuromyelitis optica, neutropenia, ocular cicatricial pemphigoid, optic neuritis, palindromic rheumatism (PR), PANDAS, paraneoplasticcerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes types I-III, polymyalgia rheumatica, polymyositis, postmyocadial infarction syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy (RSD; complex regional pain syndrome [CRPS]), relapsing polychondritis, restless leg syndrome (RLS), retroperitoneal fibrosis, rheumatic fever, rheumatoid arthritis (RA), sarcoidosis, Schmidt syndrome, scleritis, scleroderma, Sjörgren's syndrome, sperm & testicular autoimmunity, stiff person syndrome (SPS), subacute bacterial endocarditis (SBE), Susac's syndrome, sympathetic ophthalmia (SO), Takayasu arteritis, temporal arteritis/giant cell arteritis, thrombocytopenic purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo, or Vogt-Koyanagi-Harada disease. In some embodiments, the localized site of an autoimmune disease includes, for example, but is not limited to, a joint or other area with inflammation, pain or damage from rheumatoid arthritis; an area affected by juvenile dermatomyositis; psoriatic rash or a joint or other area with psoriatic inflammation; a dermal or other region with symptoms of lupus or eczema; a vascular region damaged by vasculitis; an area of myelin sheath damaged by multiple sclerosis; or a pancreatic islet damaged by type 1 diabetes.

Alternatively, protein production locally for autoimmune diseases targets the pathogenic antibodies in the disease, for example, a protein that breaks down antibodies in the vicinity (an IgG endopeptidase) or a protein that binds antibodies (a decoy of the antibody's autoimmune target).

In some embodiments, the allergic reaction includes, for example, but is not limited to, a localized allergic reaction or hypersensitivity reaction including a skin rash, hives, localized swelling (e.g., from an insect bite), or esophageal inflammation from food allergies or eosinophilic esophagitis, other enteric inflammation from food allergies or eosinophilic gastrointestinal disease, localized drug allergies when the drug treatment was local to a part of the body, or allergic conjunctivitis.

In some embodiments, the localized site of an infection or the localized site of an infectious disease includes, for example, but is not limited to, a fungal infection (e.g., aspergillus, coccidioidomycosis, tinea pedis (foot), tinea corporis (body), tinea cruris (groin), tinea capitis (scalp), and tinea unguium (nail)) , a bacterial infection (e.g., methicillin-resistant Staphylococcus aureus [MRSA], localized skin infections, abscesses, necrotizing facsciitis, pulmonary bacterial infections [e.g., pneumonia], bacterial meningitis, bacterial sinus infections, bacterial cellulitis, such as due to Staphylococcus aureus (MRSA), bacterial vaginosis, gonorrhea, chlamydia, syphilis, Clostridium difficile (C. diff), tuberculosis, cholera, botulism, tetanus, anthrax, pneumococcal pneumonia, bacterial meningitis, Lyme disease), a viral infection (e.g., varicella-zoster/herpes zoster [shingles], Herpes simplex I [e.g., cold sores/fever blisters], Herpes simplex II [genital herpes], or human papilloma virus [e.g., cervical cancer, throat cancer, esophageal cancer, mouse cancer], Epstein-Barr virus [e.g., nasopharyngeal cancer], encephalitis viruses [e.g., brain inflammation], or hepatitis viruses [e.g., liver disease; hepatitis A, hepatitis B, hepatitis C, hepatitis D, hepatitis E, hepatitis F, hepatitis G] or COVID-19), a parasitic infection (e.g., an area infected by scabies, Chagas, Hypoderma tarandi, amoebae, roundworm, Toxoplasma gondii). In some embodiments, the injury or other damage includes, for example, but is not limited to traumatic injury (e.g., resulting from an accident or violence) or chronic injury (e.g., osteoarthritis). In some embodiments, the localized site of injury comprises a muscular-skeletal injury, a neurological injury, an eye or ear injury, an internal or external wound, or a localized abscess, an area of mucosa that is affected (e.g., conjunctiva, sinuses, esophagus), or an area of skin that is affected (e.g., infection, autoimmunity). In some embodiments, the transplant or other surgical site includes, for example, but is not limited to, the site and/or its local environment or surroundings of an organ, corneal, skin, limb, face, or other transplant, or a surgical site and/or its local environment or surroundings, for, e.g., but not limited to, treatment of surgical trauma, treatment of a condition related to the transplant or surgery, or prevention of infection. In some embodiments, the site is at or adjacent to a blood clot causing or at risk for causing a myocardial infarction, an ischemic stroke, or a pulmonary embolism. In some embodiments, the methods disclosed herein treat one or more symptoms of a disease, reaction, infection, injury, transplant, surgery, or blood clot. In some embodiments, the methods disclosed herein treat a combination thereof.

In some embodiments, methods of treating described herein for promoting clearance of or alleviating localized symptoms of the autoimmune disease, allergic reaction, hypersensitivity reaction, infection or infectious disease; for facilitating healing and/or preventing or inhibiting infection or rejection of a localized site of an injury or other damage, a transplant or other surgical site; for reducing or eliminating a blood clot causing or at risk for causing a myocardial infarction, an ischemic stroke, or a pulmonary embolism; or for alleviating localized symptoms thereof; or for a combination thereof.

In some embodiments, the methods further comprises a step of administering activated T cells to said subject. Methods of preparing T cells are known in the art. In some embodiments, these cells may be administered prior to or after administering the treated leukocytes. In some embodiments, T cells are administered by intravenous (i.v., IV) injection.

Treatment of the subject the methods herein may also be used in conjunction with other known treatments. In a non-limiting example, when the disease or medical condition comprises a blood clot causing or at risk for causing a myocardial infarction, an ischemic stroke, or a pulmonary embolism, and the treatment may also include angioplasty or another clot removal treatment. Other examples of treatment include various other immunotherapies.

In some embodiments, regulating the immune response increases proliferation of cytotoxic T cells; increases proliferation of helper T cells; maintains the population of helper T cells at the site of said localized site of an autoimmune disease, a localized infection or an infectious disease, an injury or other damage, a transplant or other surgical site; activated cytotoxic T cells at the site of said localized site of an autoimmune disease, a localized infection or an infectious disease, an injury or other damage, a transplant or other surgical site, a blood clot causing or at risk for causing a myocardial infarction, an ischemic stroke, or a pulmonary embolism, or any combination thereof.

In some embodiments, methods of treating described herein reduce the size of the tumor, eliminate said tumor, slow the growth or regrowth of the tumor, or prolong survival of said subject, or any combination thereof. In some embodiments, treating reduces or eliminates inflammation or another symptom of the autoimmune-targeted or symptomatic focus of an autoimmune disease, prolongs survival of the subject, or any combination thereof; reduces or eliminates inflammation or another symptom of allergic reaction or hypersensitivity reaction at the reactive focus of an allergic reaction or hypersensitivity reaction, prolongs survival of the subject, or any combination thereof; reduces or eliminates infection or symptoms at the focus of infection or symptoms of a localized infection or infectious disease, prolongs survival of the subject, or any combination thereof; reduces, eliminates, inhibits or prevents structural, organ, tissue, or cell damage, inflammation, infection, or another symptom at a site of injury or a site of chronic damage, improves structural, organ, tissue, or cell function at a site of injury or a site of chronic damage, improves mobility of the subject, prolongs survival of the subject, or any combination thereof; reduces, eliminates, inhibits, or prevents structural, organ, tissue, or cell damage, inflammation, infection, or another symptom at a surgical site, improves structural, organ, tissue, or cell function at a surgical site, improves mobility of the subject, prolongs survival of the subject, or any combination thereof; reduces, eliminates, inhibits or prevents transplanted organ, tissue, or cell damage or rejection, inflammation, infection or another symptom at a transplant site, improves mobility of the subject, prolongs survival of a transplanted organ, tissue, or cell, prolongs survival of the subject, or any combination thereof; or reduces or eliminates a blood clot causing or at risk for causing a myocardial infarction, an ischemic stroke, or a pulmonary embolism in the subject, improves function or survival of a heart, brain, or lung organ, tissue, or cell in the subject, reduces damage to a heart, brain, or lung organ, tissue, or cell in the subject, prolongs survival of a heart, brain, or lung organ, tissue, or cell in the subject, prolongs survival of the subject, or any combination thereof.

### Tumors

In one embodiment, the tumor or the cancer for which enhancement of immunity or expansion of CTLS is provided to treat a tumor or cancer. Non-limiting examples include esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CB FB -MYH11; adult acute myeloid leukemia with t(16;16)(p13.1 ;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In another related aspect, the tumor or cancer comprises a metastasis of a tumor or cancer. In some embodiments, a solid tumor treated using a method described herein, originated as a blood tumor or diffuse tumor.

### Alternate Embodiment of The Invention: Suppressing Tregs

When activating T cells to fight, in one embodiment, solid tumors, it is essential that the microparticles do not induce the formation of regulatory T cells. This type of T cell is suppressive of adaptive immune responses through a variety of mechanisms. Overall, regulatory T cells are a major bane of therapy against cancers. To address this problem, the membrane particles of the invention may be provided with the ability to suppress the development of induced Tregs. In one embodiment, particles comprise and secrete an inhibitor of TGF-β, and the effect is that Tregs are suppressed while “conventional” effector T cells against the tumor antigens are promoted.

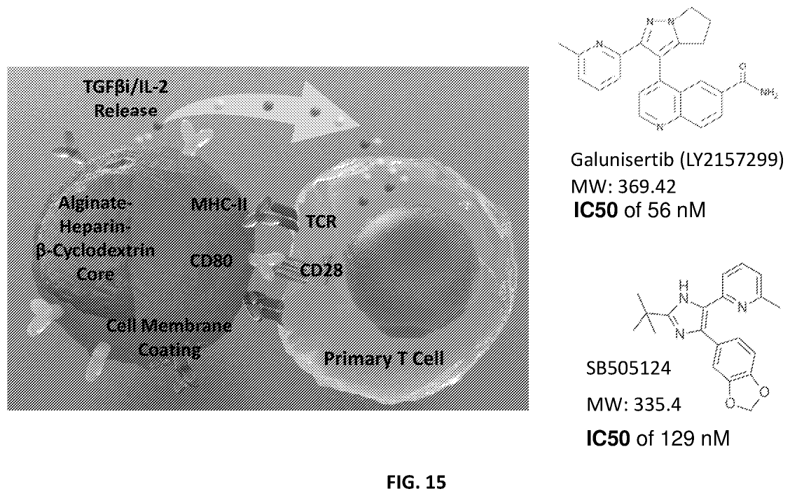

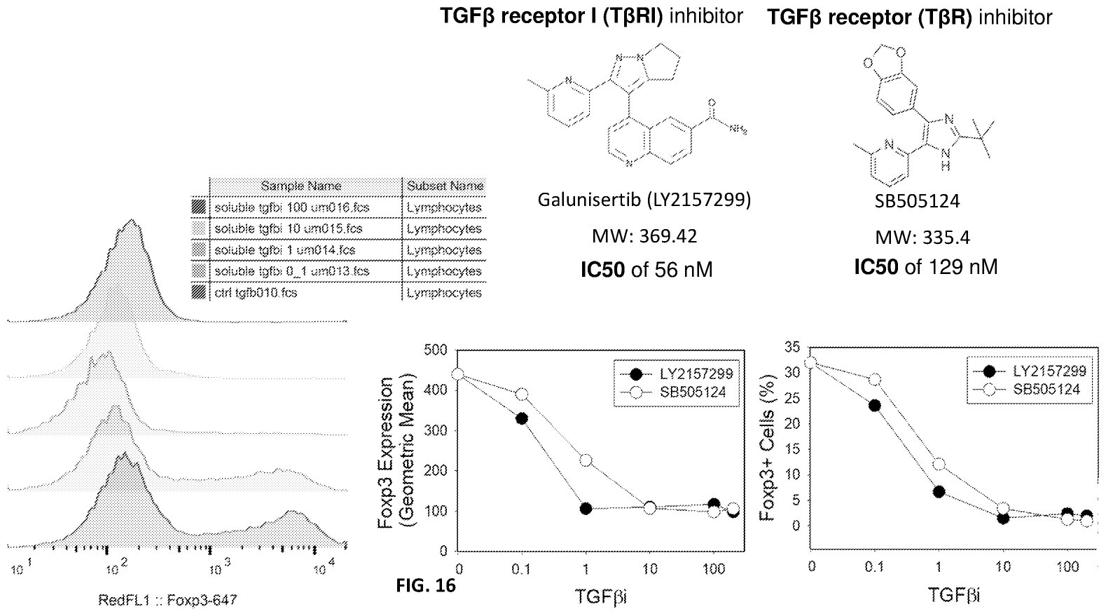

Thus, in one embodiment, an inhibitor or blocker of TGF-β is included or incorporated into the microparticles of the invention. A TGF-β inhibitor (TGF-βi) such as a TGF-β receptor inhibitor may be used. Non-limiting examples include galinusertib (LY2157299) or SB505124. As shown in FIG. 16, these compounds may be provided to block the induction of Tregs. In studies shown in FIG. 15, two TGFβ inhibitors, galunisertib (LY2157299) and SB505124 were evaluated to determine which one is more effective on suppressing Treg formation. Decreasing Foxp3 expression and having a fewer number of Foxp3+ cells at the same concentration of TGF-β inhibitors showed that galunisertib (LY2157299) provides stronger Treg suppression. This TGFβ inhibitor used for further studies as described in FIGS. 16-20.

### Alternate Embodiment of the Invention: Inducing Tregs for Treating Autoimmunity

In another embodiment, rather than activating T cells to respond to tumor antigens and kill tumor cells, an alternate form of microparticles are described herein that activate T cells in response to self antigens. This approach, when combined with chemical augmentation of T cells, can elicit regulatory T cells. Regulatory T cells can be delivered to mitigate autoimmune diseases. It is not often (ever) known what the self antigen is for autoimmune diseases, as there are thousands of unique proteins that may be specific for a particular tissue that is under autoimmune attack. Thus, it has been difficult to develop a strategy for tolerizing T cells to the right autoantigen.

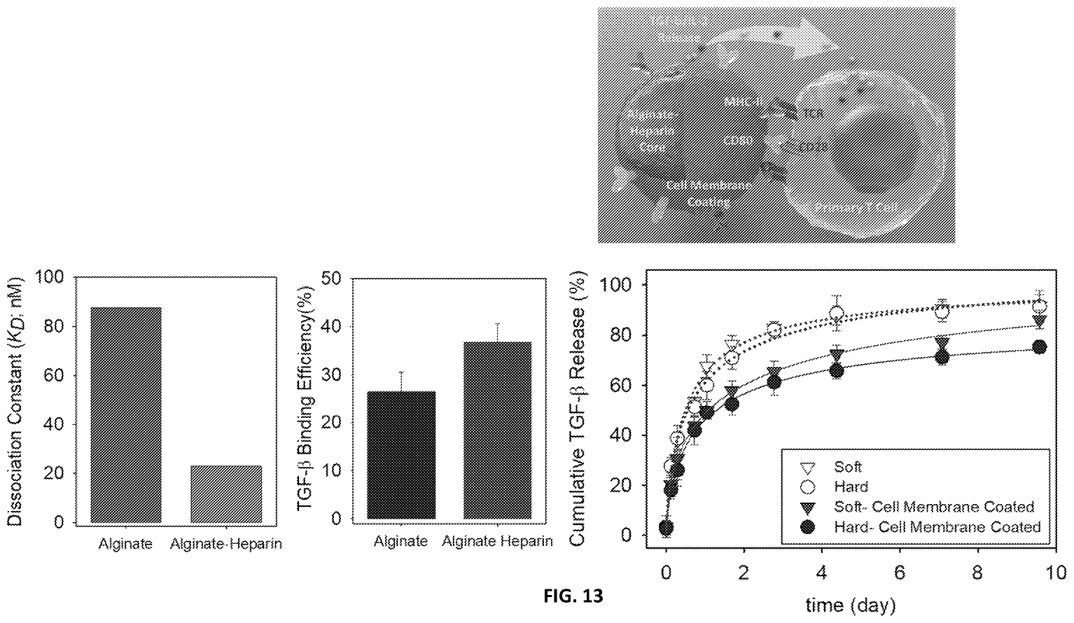

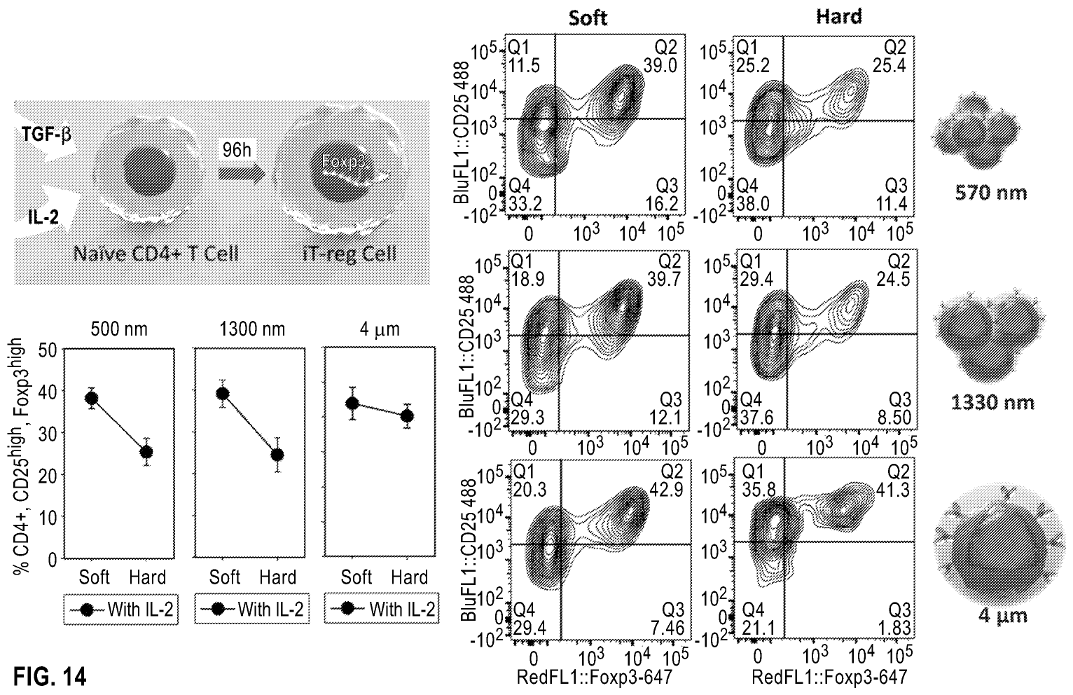

In this embodiment, self-antigens from tissues undergoing autoimmune attack can be prepared by coating particles with membranes thereof. In this case the particles are engineered to release TGF-β and IL-2, which together promote regulatory T cell development (so called induced regulatory T cells). This is depicted in FIGS. 13-14. The microparticles are used in the same manner as described above, by exposing peripheral blood mononuclear cells obtained from the patient's whole blood or pheresis to the microparticles ex vivo, then separation of the particles and infusion of the resulting cell population into the patient. An in-line or leukapheresis device may incorporate the microparticles of the invention.

In some embodiments, the methods are used for the treatment of vertebrate organisms. In some embodiments, the methods are used for the treatment of homeothermic vertebrate organisms (e.g., mammals and birds). In some embodiments, the methods are used for the treatment of human or non-human mammals.

The following examples are presented in order to more fully illustrate the preferred embodiments of the invention. It should in no way be construed, however, as limiting the broad scope of the invention.

### EXAMPLES

### Example 1

### Production of Microparticles and Methods of Use

A microparticle is constructed comprising an antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A sample of cells or tissue of interest is obtained and/or grown in vitro, and membranes, comprising the antigen of interest, are isolated from the cells or tissue of interest. A sample of antigen presenting cells (APCs) is obtained, and membranes, comprising the costimulatory components, are isolated from the APCs, which are optionally stimulated in vitro prior to isolating of the membranes. The microparticle optionally includes an immunoregulatory compound and/or a compound that regulates inductions of regulatory T cells (Tregs).

Leukocytes are obtained (e.g., via whole blood from a subject or following pheresis) and exposed in vitro or ex vivo to the microparticles to generate an activated cytotoxic T cell population specific for the antigen of interest. The activated cytotoxic T cells are then reinfused into the subject.

### Example 2

### Production of Immunostimulatory Microparticles and Methods of Use

A microparticle is constructed comprising an antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A sample of cells or tissue of interest is obtained and/or grown in vitro, and membranes, comprising the antigen of interest, are isolated from the cells or tissue of interest. A sample of antigen presenting cells (APCs) is obtained, and membranes, comprising the costimulatory components, are isolated from the APCs, which are optionally stimulated in vitro prior to isolating of the membranes.

The microparticle further comprises an immunostimulatory compound (e.g., a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid) and/or a compound that suppresses induction of Tregs (e.g., a TGF-β inhibitor).

Leukocytes are obtained (e.g., via whole blood from a subject or following pheresis) and exposed in vitro or ex vivo to the microparticles to generate an activated cytotoxic T cell population specific for the antigen of interest. The activated cytotoxic T cells are then reinfused into the subject, where they stimulate the desired immune response.

### Example 3

### Production of Immunosuppression Microparticles and Methods of Use

A microparticle is constructed comprising an antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A sample of cells or tissue of interest is obtained and/or grown in vitro, and membranes, comprising the antigen of interest, are isolated from the cells or tissue of interest. A sample of antigen presenting cells (APCs) is obtained, and membranes, comprising the costimulatory components, are isolated from the APCs, which are optionally stimulated in vitro prior to isolating of the membranes.

The microparticle further comprises an immunosuppression compound (e.g., a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid) and/or a compound that induces Tregs (e.g., a TGF-β or an activator thereof).

Leukocytes are obtained (e.g., via whole blood from a subject or following pheresis) and exposed in vitro or ex vivo to the microparticles to generate an activated cytotoxic T cell population specific for the antigen of interest. The activated cytotoxic T cells are then reinfused into the subject, where they suppress the desired immune response.

### Example 4

### Preparation of an Activated Cytotoxic T Cell Population

Here, the microparticles are prepared as described above. The antigen is obtained from a sample from the patient or other subject, as described above, and leukocytes are obtained from whole blood or pheresis, e.g., of blood from a patient or other subject. The leukocytes are exposed in vitro or ex vivo to the microparticles, which are then separated from the leukocytes, e.g., by magnetic separation or by other methods of separation known in the art. The treated leukocytes are then reinfused into the patient or other subject.

This method is particularly useful for targeting inoperable or inaccessible conditions, such as an inoperable tumor.

### Example 5

### Treatment of Cancerous, Pre-Cancerous, and Non-Cancerous Tumors with Microparticles

A microparticle is constructed comprising a tumor or cancer-specific antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A tumor or a sample of tumor or cancer cells or tissue of interest is obtained (e.g., obtained via surgery or biopsy or from a sample) and/or grown in vitro, and membranes, comprising the tumor or cancer antigen of interest, are isolated from the tumor or from the sample of tumor or cancer cells or tissue. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunostimulatory compound (e.g., a cytokine [e.g., IL-2, IL-12, IL-15], a growth factor, a chemokine [e.g., CCL21], a therapeutic or diagnostic antibody or fragment thereof [e.g., anti-CD3, anti-CD28], an antigen-binding protein, a Fc fusion protein, an anticoagulant, an enzyme, a hormone, a thrombolytic, a peptide, an oligonucleotide, or a nucleic acid) and/or a compound that suppresses induction of Tregs (e.g., a TGF-β inhibitor [e.g., TGFβi]). The T cell immunostimulatory compound and/or the compound that suppresses induction of Tregs are selected for the treatment of a cancerous, pre-cancerous, or non-cancerous tumor, or for the alleviation of localized symptoms, or combinations thereof, in a subject. The T cell immunostimulatory compound and/or the compound that suppresses induction of Tregs acts in concert with the personalized activated cytotoxic T cells to enhance a desired immune response for the treatment or reduction in size of the cancerous, pre-cancerous, or non-cancerous tumor. The combination of the personalized activated cytotoxic T cells, along with the T cell immunostimulatory compound and/or the compound that suppresses induction of Tregs is selected, e.g., to inhibit cell division and/or growth (e.g., a growth factor inhibitor), to inhibit angiogenesis (e.g., an angiogenic factor inhibitor), to promote cell death (e.g., an apoptosis-promoting cytokine or other protein of interest), or to regulate an immune response (e.g., increasing proliferation of cytotoxic T cells, increasing proliferation of helper T cells, maintaining the population of helper T cells, activating cytotoxic T cells, or a combination thereof), in the vicinity of the tumor. Where the tumor is cancerous or pre-cancerous (e.g., a growth comprising cells with at least one pre-cancerous mutation), the T cell immunostimulatory compound and/or the compound that suppresses induction of Tregs may be selected based on the type(s) of cells comprising the tumor and, e.g., any cell surface proteins specific to the cancerous or pre-cancerous cells as compared with neighboring healthy tissue.

The personalized activated T cells, optionally in concert with a T cell immunostimulatory compound and/or a compound that suppresses induction of Tregs at, adjacent to, or near the site of the focus of interest treat(s) a localized environment comprising the tumor or cancer within the subject.

### Example 6

### Treatment of an Autoimmune-Targeted Focus or of a Symptomatic Focus of an Autoimmune Disease with Microparticles

A microparticle is constructed comprising a self antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A sample of a cell, a tissue, or an organ targeted by an autoimmune disease or undergoing autoimmune attack is obtained (e.g., obtained via surgery or biopsy or from a sample) and/or grown in vitro, and membranes, comprising the self antigen of interest, are isolated from the sample of a cell, a tissue, or an organ targeted by an autoimmune disease or undergoing autoimmune attack. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunosuppression compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], LINK, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid) and/or a compound that induces Tregs (e.g., a TGF-β or an activator thereof). The T cell immunosuppression compound and/or the compound that induces Tregs are selected for the treatment of a an autoimmune disease (e.g., rheumatoid arthritis, psoriasis, eczema, multiple sclerosis), or for the alleviation of localized symptoms, or combinations thereof, in a subject. The T cell immunostimulatory compound and/or the compound that suppresses induction of Tregs acts in concert with the personalized activated cytotoxic T cells to enhance a desired immune response for the treatment of the autoimmune disease and/or alleviation of the symptoms thereof. The combination of the personalized activated cytotoxic T cells, along with the T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to inhibit or promote (as needed) cell death (e.g., an apoptosis-promoting cytokine or other protein of interest), or to regulate an immune response (e.g., decreasing proliferation of cytotoxic T cells, decreasing proliferation of helper T cells, reducing cytotoxic T cells, or a combination thereof, as well as increasing recognition of self), in the vicinity of the autoimmune-targeted or symptomatic focus of the autoimmune disease.

The personalized activated T cells, optionally in concert with a T cell immunostimulatory compound and/or a compound that suppresses induction of Tregs at, adjacent to, or near the site of the focus of interest treat(s) a localized environment comprising an autoimmune-targeted focus or a symptomatic focus of autoimmune disease within the subject.

### Example 7

### Treatment of an Allergic Reaction or a Reactive Focus of an Allergic Reaction with Microparticles

A microparticle is constructed comprising an IgE receptor-specific antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A sample of a mast cell or a basophil generated by an allergic reaction or hypersensitivity reaction is obtained (e.g., obtained via surgery or biopsy or from a sample) and/or grown in vitro, and membranes, comprising the IgE receptor-specific antigen of interest, are isolated from the sample of a mast cell or basophil. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunosuppression compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], LINK, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid) and/or a compound that induces Tregs (e.g., a TGF-β or an activator thereof). The T cell immunosuppression compound and/or the compound that induces Tregs are selected for the treatment of a reactive focus of an allergic reaction, hypersensitivity reaction, or for the alleviation of localized symptoms, or combinations thereof, in a subject. Non-limiting examples of a reactive focus of an allergic reaction or hypersensitivity reaction in a subject include a skin rash, a hive or hives, or a localized swelling (e.g., from an insect or other bite). The combination of the personalized activated cytotoxic T cells, along with the T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to inhibit or promote (as needed) cell death (e.g., an apoptosis-promoting cytokine or other protein of interest), or to regulate an immune response (e.g., decreasing production or accumulation of histamine, increasing proliferation of cytotoxic T cells, increasing proliferation of helper T cells, maintaining the population of helper T cells, activating cytotoxic T cells, or a combination thereof), in the vicinity of the reactive focus of the allergic reaction or hypersensitivity reaction.

The personalized activated T cells, optionally in concert with a T cell immunostimulatory compound and/or a compound that suppresses induction of Tregs at, adjacent to, or near the site of the focus of interest treat(s) a localized environment comprising the cells, tissues, or organs targeted by an allergic reaction or hypersensitivity reaction or a reactive focus of an allergic reaction or hypersensitivity reaction within the subject.

### Example 8

### Treatment of an Infection or Symptoms of a Localized Infection or an Infectious Disease with Microparticles

A microparticle is constructed comprising a pathogen-specific antigen and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A cell of a pathogen or a viral envelope or a viral capsid of interest or a host cell comprising a pathogen or a viral envelope or a viral capsid is obtained (e.g., obtained via surgery or biopsy or from a sample) and/or grown in vitro, and membranes, comprising the pathogen-specific antigen of interest, are isolated from or from the cell of a pathogen or a viral envelope or a viral capsid of interest or host cell comprising the viral envelope or viral capsid. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunostimulatory compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], LINK a therapeutic or diagnostic antibody or fragment thereof [e.g., anti-CD3, anti-CD28], an antigen-binding protein, a Fc fusion protein, an anticoagulant, an enzyme, a hormone, a thrombolytic, a peptide, an oligonucleotide, or a nucleic acid) and/or a compound that suppresses induction of Tregs (e.g., a TGF-β inhibitor [e.g., TGFβi]). The T cell immunostimulatory compound and/or the compound that suppresses induction of Tregs are selected for the treatment of an infection or symptoms of a localized infection or an infectious disease, or for the alleviation of localized symptoms, or combinations thereof, in a subject. The T cell immunostimulatory compound and/or the compound that suppresses induction of Tregs acts in concert with the personalized activated cytotoxic T cells to enhance a desired immune response for the treatment or reduction in the size/amount/severity of the infection or symptoms of a localized infection or an infectious disease. If, as a non-limiting example, the subject has fungal infection (e.g., tinea pedis (foot), tinea corporis (body), tinea cruris (groin), tinea capitis (scalp), and tinea unguium (nail), aspergillus, coccidioidomycosis), a bacterial infection (e.g., methicillin-resistant Staphylococcus aureus [MRSA], localized skin infections, abscesses, necrotizing facsciitis, pulmonary bacterial infections [e.g., pneumonia], bacterial meningitis, bacterial sinus infections, bacterial cellulitis, such as due to Staphylococcus aureus (MRSA), bacterial vaginosis, gonorrhea, chlamydia, syphilis, Clostridium difficile (C. diff), tuberculosis, cholera, botulism, tetanus, anthrax, pneumococcal pneumonia, bacterial meningitis, Lyme disease), a viral infection (e.g., a shingles rash from varicella-zoster/herpes zoster; a cold sore/fever blister from, e.g., Herpes simplex I; a genital wart or blister from, e.g., Herpes simplex II, or COVID-19), a parasitic infection (e.g., an area infected by scabies, Chagas, Hypoderma tarandi, an amoeba, a roundworm, Toxoplasma gondii), or a localized abscess, an area of mucosa that is affected (e.g., conjunctiva, sinuses, esophagus), or an area of skin that is affected (e.g., infection, autoimmunity), the T cell immunostimulatory compound and/or the compound that suppresses induction of Tregs treatment is administered either to treat the infection (e.g., an wound or surgical site infected with MRSA), to contain it or reduce its spread within the subject, to reduce its transmissibility to other individuals (Herpes simplex I or Herpes simplex II), or to reduce a symptom of the infection at the focus of symptoms (e.g., pain associated with an outbreak of shingles). The combination of the personalized activated cytotoxic T cells, along with the T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the focus of infection or symptoms of a localized infection or an infectious disease.

The personalized activated T cells, optionally in concert with a T cell immunostimulatory compound and/or a compound that suppresses induction of Tregs at, adjacent to, or near the site of the focus of interest treat(s) a localized environment comprising the an infection or symptoms of a localized infection or an infectious disease within the subject.

### Example 9

### Treatment of an Injury or a Site of Chronic Damage with Microparticles

A microparticle is constructed comprising an antigen specific to an organ, tissue, or cell of interest and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A damaged cell, a damaged tissue, or a cell or tissue from a damaged organ due an injury (e.g., trauma, chemical) or of a site of chronic damage (e.g., osteoarthritis, type 1 diabetes, rheumatoid arthritis, lupus) of interest is obtained (e.g., obtained via surgery or biopsy or from a sample) and/or grown in vitro, and membranes, comprising the antigen specific to the organ, tissue, or cell of interest, are isolated from the damaged cell, damaged tissue, or cell or tissue from a damaged organ. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunostimulatory compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], LINK, a therapeutic or diagnostic antibody or fragment thereof [e.g., anti-CD3, anti-CD28], an antigen-binding protein, a Fc fusion protein, an anticoagulant, an enzyme, a hormone, a thrombolytic, a peptide, an oligonucleotide, or a nucleic acid) and/or a compound that suppresses induction of Tregs (e.g., a TGF-β inhibitor [e.g., TGFβi]). The T cell immunostimulatory compound and/or the compound that suppresses induction of Tregs are selected for the treatment of an injury (e.g., trauma, chemical) or of a site of chronic damage (e.g., osteoarthritis, type 1 diabetes, rheumatoid arthritis, lupus) in the subject, or for the alleviation of localized symptoms, or combinations thereof, in a subject. The T cell immunostimulatory compound and/or the compound that suppresses induction of Tregs acts in concert with the personalized activated cytotoxic T cells to enhance a desired immune response for the treatment or reduction in the size/amount/severity of the focus of infection or symptoms of an injury or a site of chronic damage, in the subject. If, as a non-limiting example, the subject has fungal infection (e.g., tinea pedis (foot), tinea corporis (body), tinea cruris (groin), tinea capitis (scalp), and tinea unguium (nail), aspergillus, coccidioidomycosis), a bacterial infection (e.g., methicillin-resistant Staphylococcus aureus [MRSA], localized skin infections, abscesses, necrotizing facsciitis, pulmonary bacterial infections [e.g., pneumonia], bacterial meningitis, bacterial sinus infections, bacterial cellulitis, such as due to Staphylococcus aureus (MRSA), bacterial vaginosis, gonorrhea, chlamydia, syphilis, Clostridium difficile (C. diff), tuberculosis, cholera, botulism, tetanus, anthrax, pneumococcal pneumonia, bacterial meningitis, Lyme disease), a viral infection (e.g., a shingles rash from varicella-zoster/herpes zoster; a cold sore/fever blister from, e.g., Herpes simplex I; a genital wart or blister from, e.g., Herpes simplex II, or COVID-19), a parasitic infection (e.g., an area infected by scabies, Chagas, Hypoderma tarandi, an amoeba, a roundworm, Toxoplasma gondii), or a localized abscess, an area of mucosa that is affected (e.g., conjunctiva, sinuses, esophagus), or an area of skin that is affected (e.g., infection, autoimmunity), the T cell immunostimulatory compound and/or the compound that suppresses induction of Tregs treatment is either to treat, reduce, or alleviate the injury (e.g., to promote repair, to promote vascularization), to prevent infection or further damage (e.g., fungal, bacterial, viral, or parasitic infection; neuropathy; muscle wasting), or to reduce a symptom of the injury or of the chronic damage (e.g., pain, inflammation). The combination of the personalized activated cytotoxic T cells, along with the T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the injury or the site of chronic damage.

The personalized activated T cells, optionally in concert with a T cell immunostimulatory compound and/or a compound that suppresses induction of Tregs at, adjacent to, or near the site of the focus of interest treat(s) a localized environment comprising the injury or site of chronic damage within the subject.

### Example 10

### Treatment of a Surgical Site with Microparticles

A microparticle is constructed comprising an antigen specific to an organ, tissue, or cell of interest and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A cell, a tissue, or a cell or tissue from an organ, or a cell, tissue, or organ sample damaged due to surgery is obtained (e.g., obtained via surgery or biopsy or from a sample) and/or grown in vitro, and membranes, comprising the antigen specific to the organ, tissue, or cell of interest, are isolated. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunostimulatory compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], LINK, a therapeutic or diagnostic antibody or fragment thereof [e.g., anti-CD3, anti-CD28], an antigen-binding protein, a Fc fusion protein, an anticoagulant, an enzyme, a hormone, a thrombolytic, a peptide, an oligonucleotide, or a nucleic acid) and/or a compound that suppresses induction of Tregs (e.g., a TGF-β inhibitor [e.g., TGFβi]). The T cell immunostimulatory compound and/or the compound that suppresses induction of Tregs are selected for the treatment of a surgical site in the subject, or for the alleviation of localized symptoms, or combinations thereof, in a subject. The T cell immunostimulatory compound and/or the compound that suppresses induction of Tregs acts in concert with the personalized activated cytotoxic T cells to enhance a desired immune response either to treat, reduce, or alleviate the effects of surgery (e.g., to promote repair, to promote vascularization), to prevent infection or further damage (e.g., fungal, bacterial, viral, or parasitic infection; neuropathy; muscle wasting), or to reduce a symptom of the effects of surgery (e.g., pain, inflammation), in the subject. The combination acts in concert with other proteins or cells to enhance a desired immune response for the treatment or reduction in the size/amount/severity of the surgical site and type of surgery, as well as of one or more localized symptoms of the associated effects of surgery. The combination of the personalized activated cytotoxic T cells, along with the T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the surgical site.

The personalized activated T cells, optionally in concert with a T cell immunostimulatory compound and/or a compound that suppresses induction of Tregs at, adjacent to, or near the site of the focus of interest treat(s) a localized environment comprising the surgical site within the subject.

### Example 11

### Treatment of a Transplant Site Associated with a Transplanted Organ, Tissue, or Cells with Microparticles

A microparticle is constructed comprising an antigen specific to a transplanted organ, tissue, or cell of interest and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. A sample of a transplanted cell, tissue, or organ is obtained (e.g., obtained via surgery or biopsy or from a sample) and/or grown in vitro, and membranes, comprising the antigen specific to the transplanted cell, tissue, or organ of interest, are isolated from the sample of a transplanted cell, tissue, or organ. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunosuppression compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid) and/or a compound that induces Tregs (e.g., a TGF-β or an activator thereof). The T cell immunosuppression compound and/or the compound that induces Tregs are selected for the treatment associated with a transplanted organ, tissue, or cells, either to treat, reduce, or alleviate the surgery related to the transplant (e.g., to promote repair, to promote vascularization), to prevent infection or damage (e.g., fungal, bacterial, viral, or parasitic infection; neuropathy; muscle wasting), to reduce the likelihood of rejection, or to reduce a symptom of the transplant or surgery related thereto (e.g., pain, inflammation). The T cell immunostimulatory compound and/or the compound that suppresses induction of Tregs acts in concert with the personalized activated cytotoxic T cells to reduce the immune response for the treatment of the transplant site associated with a transplanted organ, tissue, or cells. The combination of the personalized activated cytotoxic T cells, along with the T cell immunostimulatory compound and/or the compound that suppresses induction of Tregs is selected, e.g., to inhibit or promote (as needed) cell division and/or growth (e.g., a growth factor inhibitor), to inhibit inflammation (e.g., anti-inflammatory), to promote analgesic activity, to inhibit or promote (as needed) cell death (e.g., an apoptosis-promoting cytokine or other protein of interest), or to regulate an immune response, such as suppression of rejection (e.g., decreasing proliferation of cytotoxic T cells, decreasing proliferation of helper T cells, decreasing cytotoxic T cells, or a combination thereof), in the vicinity of the injury or the site of chronic damage.

The personalized activated T cells, optionally in concert with a T cell immunostimulatory compound and/or a compound that suppresses induction of Tregs at, adjacent to, or near the site of the focus of interest treat(s) a localized environment comprising the transplant site associated with a transplanted organ, tissue, or cells within the subject, thereby reducing the likelihood of rejection and/or one or more of its symptoms or effects, as well as the symptoms or effects of the transplant surgery.

### Example 12

### Treatment of a Blood Clot Causing or at Risk for Causing a Myocardial Infarction, an Ischemic Stroke, or a Pulmonary Embolism with Microparticles

A microparticle is constructed comprising a macrophage-specific antigen; an erythrocyte-specific antigen; a platelet-specific antigen; a platelet factor 5-specific antigen; a fibrinogen-specific antigen; a stem cell- or progenitor cell-specific antigen; a lymphocyte-specific antigen; a monocyte-specific antigen; an immune cell-specific antigen; or a stromal cell-specific antigen; or a combination thereof and a costimulatory component derived from an antigen presenting cell. A microparticle comprising a biocompatible polymer (e.g., alginate, hyaluronic acid, or chitosan), optionally crosslinked, is provided. The microparticle optionally comprises heparin. An erythrocyte or a platelet is obtained (e.g., obtained via surgery or biopsy or from a sample) and/or grown in vitro, and membranes, comprising the macrophage-specific antigen; erythrocyte-specific antigen; platelet-specific antigen; platelet factor 5-specific antigen; fibrinogen-specific antigen; a stem cell- or progenitor cell-specific antigen; a lymphocyte-specific antigen; a monocyte-specific antigen; an immune cell-specific antigen; or a stromal cell-specific antigen; or a combination thereof, are isolated from the erythrocyte or platelet. A sample of antigen presenting cells (APCs) is obtained, and membranes, comprising the costimulatory components, are isolated from the APCs, which are optionally stimulated in vitro prior to isolating of the membranes.

Leukocytes are obtained (e.g., via whole blood from a subject or following pheresis) and exposed in vitro or ex vivo to the microparticles to generate a personalized activated cytotoxic T cell population specific for the antigen of interest. The personalized activated cytotoxic T cells are then reinfused into the subject, where they stimulate the desired immune response.

Optionally, the microparticle further comprises an immunoregulatory compound (e.g., a cytokine [e.g., IL-2, IL-4, IL-6, IL-7, IL-10, IL-12, IL-15], a growth factor, a chemokine [e.g., CCL19, CCL21, CXCL4, CXCL9, CXCL10, CCL5], a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid) and/or a compound that regulates the induction of Tregs). The compound that regulates T cell immune response and/or the compound that regulates induction of Tregs (e.g., a cytokine, a thrombolytic, or another protein of interest) are selected for the treatment of selected for the treatment of a blood clot causing or at risk for causing a myocardial infarction, an ischemic stroke, or a pulmonary embolism in the subject, or for the alleviation of localized symptoms, or combinations thereof, in a subject. In a non-limiting example, thrombolytic (“clot buster”) treatment is administered at or adjacent to the blood clot to break up, reduce, or eliminate the blood clot in order to treat or prevent infarction of a blood vessel and thereby to treat or prevent, e.g., a myocardial infarction (heart attack), an ischemic stroke, or a pulmonary embolism. Non-limiting examples of thrombolytics include tissue plasminogen activator (tPA), tenecteplase, alteplase, urokinase, reteplase, and streptokinase.

The T cell immunoregulatory compound and/or the compound that regulates induction of Tregs are selected for the treatment associated with a transplanted organ, tissue, or cells, either to treat, reduce, or alleviate the effects of surgery (e.g., to promote repair, to promote vascularization), to prevent infection or further damage (e.g., fungal, bacterial, viral, or parasitic infection; neuropathy; muscle wasting), or to reduce a symptom of the effects of surgery (e.g., pain, inflammation). It is noted that the location of the blood clot may not be in the heart, the brain, or a lung at the time of treatment, but rather in some other part of the subject's body (e.g., the lower limbs and extremities; the carotid artery; the site of an injury, surgery, or a transplant; or elsewhere). The T cell immunoregulatory compound and/or the compound that regulates induction of Tregs acts in concert with the personalized activated cytotoxic T cells to enhance a desired response for the treatment of a blood clot. The combination of the personalized activated cytotoxic T cells, along with the T cell immunoregulatory compound and/or the compound that regulates induction of Tregs is selected, e.g., to inhibit angiogenesis, to promote reduction or elimination of clotting, to inhibit inflammation (e.g., anti-inflammatory), to promote analgesic activity, to inhibit or promote (as needed) cell death (e.g., an apoptosis-promoting cytokine or other protein of interest), or to regulate an immune response (e.g., increasing proliferation of cytotoxic T cells, increasing proliferation of helper T cells, maintaining the population of helper T cells, increasing cytotoxic T cells, or a combination thereof), in the vicinity of the blood clot.

The treatment may be administered via a guided catheter, which may facilitate access to, and treatment of, the blood clot. The treatment may be administered, in a non-limiting example, together with angioplasty (e.g., a balloon catheter) or other clot removal treatment.

The personalized activated T cells, optionally in concert with a T cell immunoregulatory compound and/or a compound that regulates induction of Tregs at, adjacent to, or near the site of the focus of interest treat(s) a localized environment comprising the blood clot within the subject.

### Materials and Methods for Examples 13-16

### Tumor Models

B16 melanoma—Syngeneic with C57B1/6 mice was used as a tumor model. The B16 strain expresses ovalbumin, which allows for expression on H2-K^{b} of the SIINFEKL (SEQ ID NO: 1) peptide that is cognate for CD8+ OT-I TCR transgenic mice, and when presented by professional APCs on I-A^{b}, the Ova 323-339 peptide that is cognate for CD4+ OT-II mice. These cells also express GFP. B16 melanoma grows fast and tests the adaptive immune response to ramp up quickly—the suppressive nature of regulatory T cells plays a major role more than CD8+ exhaustion, as does the ability of effector CD8+ T cells to gain cytotoxic capability.

Tumor size was assessed over time using a digital caliper until day 22 at which animals sacrificed and the tumor was extracted. Tumor mass was measured using a digital balance before digesting the tumor tissue for flow cytometry. Tissues were digested by incubating in collagenase and DNase I (50 micrograms (μg)/mL) at 37° C. for 15 min. (Weigelin, B. et al. ibid). These enzymes were inactivated with ethylenediaminetetraacetic acid (EDTA) (20 microliters [μL]/mL of solution). Tissues then were mechanically disaggregated and passed by a 0.7 micron (μm) cell strainer to obtain a single-cell suspension. Cells were then stained with the fluorochrome-conjugated antibodies on ice. For intracellular staining (e.g., Granzyme B), cells were permeabilized with FIX/PERM™ buffer according to manufacturer instructions (BIOLEGEND™) before staining.

As an alternative, the B16-gp33 melanoma line has also been used; it activates the CD8+ TCR transgenic p14 with its cognate peptide gp33 derived from the LCMV virus.

### Microparticles

Microparticles were generated by microfluidic droplet generation using alginate-heparin as the main component, such as shown in FIG. 2. The alginate was crosslinked using a 4-arm PEG hydrazide cross linker. Embedded in each microparticle were superparamagnetic nanoparticles as well. As shown in FIG. 2, the flow ratio in the generator can be adjusted for different droplet diameters which translate to particle sizes (and dispersities). The size distribution of the resulting microparticles is shown.

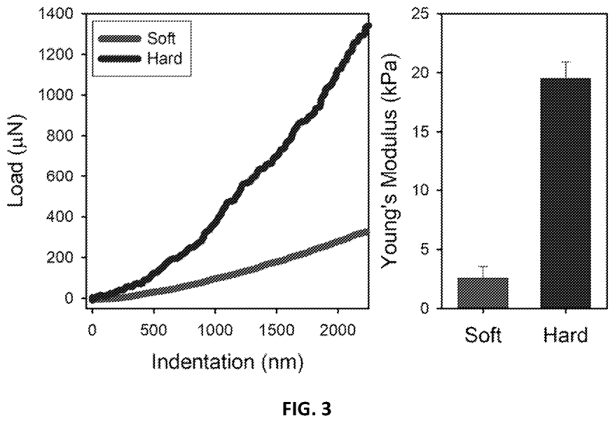

By varying the amount of crosslinker, the particles can be made mechanically soft or stiff. Recent work has suggested that altering the mechanical stiffness of substrates can influence the spreading, and consequently activation, of T cells. FIG. 3 shows the load vs. indentation relationships, and Young's modulus, of a hard and soft particle. By changing the polymer concentration (0.5-2.5 wt/v %), the 4-arm PEG hydrazide concentration (1-15 mM) and the calcium concentration (5-200 mM), the mechanical stiffness of the microparticle is tunable. Increasing the concentration any one or more of these components increases the mechanical stiffness.

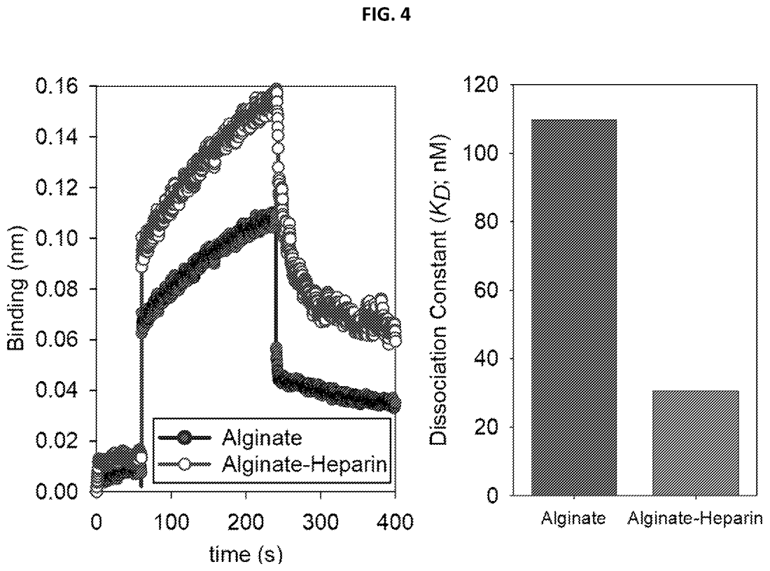

To promote T cell activation, the stimulatory cytokine IL-2 is loaded in the particles for uptake and later, passive release. FIG. 4 shows that by including heparin in the microparticle formulation, more IL-2 can be retained by the particles and released more slowly. The conjugation density can be tuned; in these studies, 0.1-2.0 nmol heparin was used per mg of alginate.

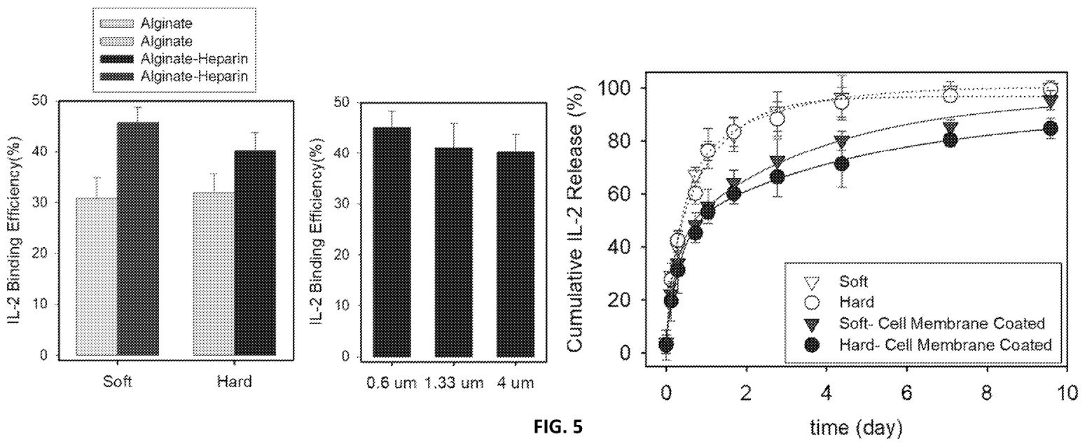

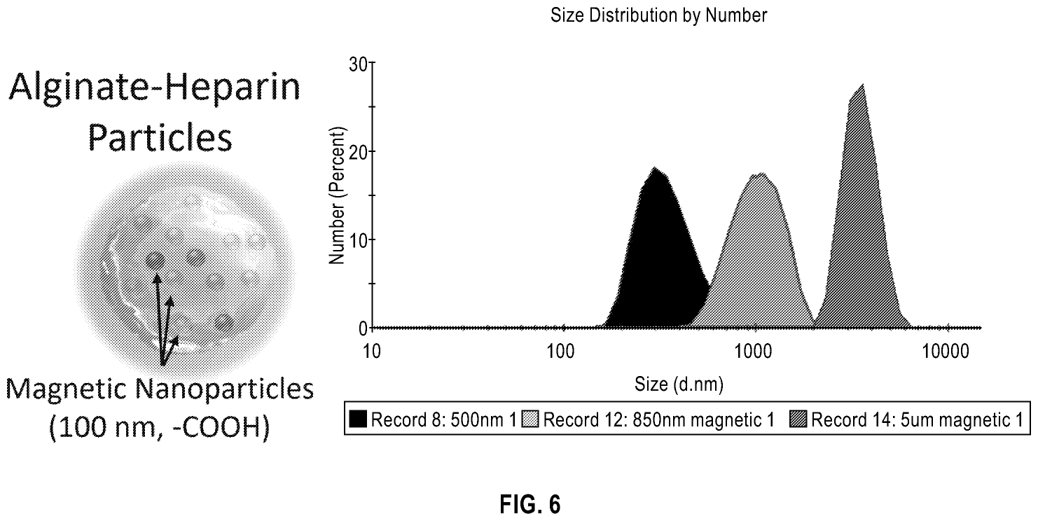

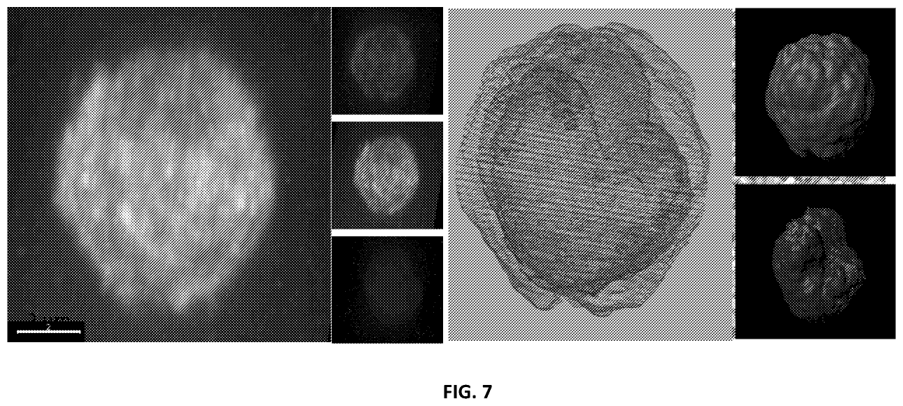

Alginate-heparin can be modified and purified before making microparticles. The dissociation constants of heparin from the alginate and alginate-heparin microparticles is shown; binding was measured at 7C under gentle shaking in PBS. In FIG. 5, the IL-2 binding efficiency to soft and hard microparticles is shown, which is not dependent on the microparticle size. The release of IL-2 over time of the different microparticles is shown in FIG. 5, and the distribution by number of magnetic nanoparticles per microparticle in FIG. 6. FIG. 7 shows B cells which have quite large nuclei and makes the cell membrane extraction difficult.

### APC Membrane Preparations

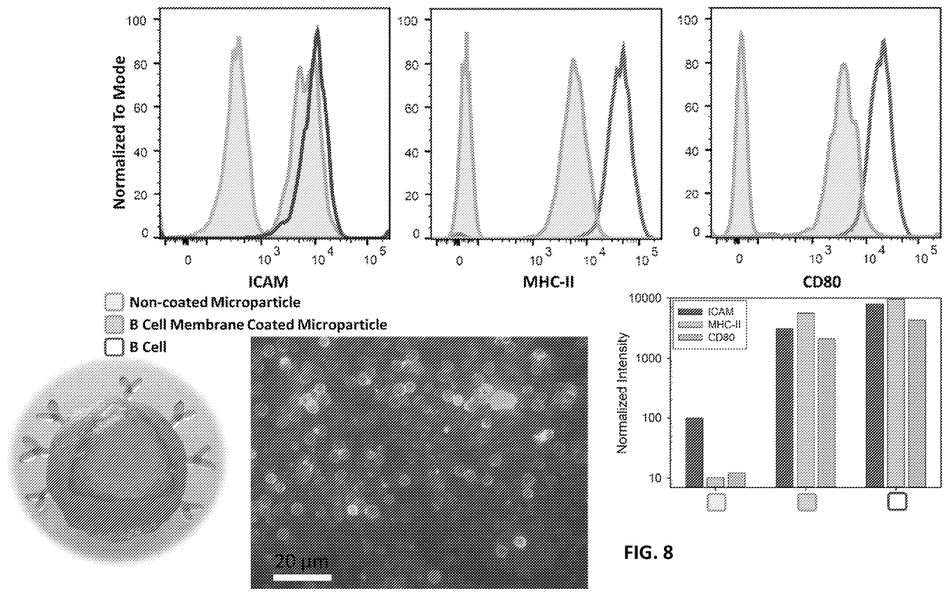

B cells were activated in vitro and lysed. LPS may be used for activation. The membranes were collected after centrifugation and incubated with the alginate-heparin microparticles (FIG. 6). To demonstrate that the particles have picked up B cell membranes, flow cytometry was conducted on the B cells (FIG. 8) and the nanoparticles (NPs) and showed that they have plentiful expression of the cell-surface adhesion molecule ICAM-1, the antigen presenting molecule MHC-II, and the costimulatory ligand B7-1 (CD80). This procedure can be used for both microparticles and nanoparticles with an overall size range of about 20 nm to about 300 microns (μm).

When the sizes of the particles were normalized to the size of the B cells, it was observed that expression of these three markers was exactly comparable to the B cells themselves, showing that there was no loss of density of surface molecules upon transfer to the NPs (FIG. 8).

### Example 13

### Evaluation of Stimulation of Proliferation by Microparticles

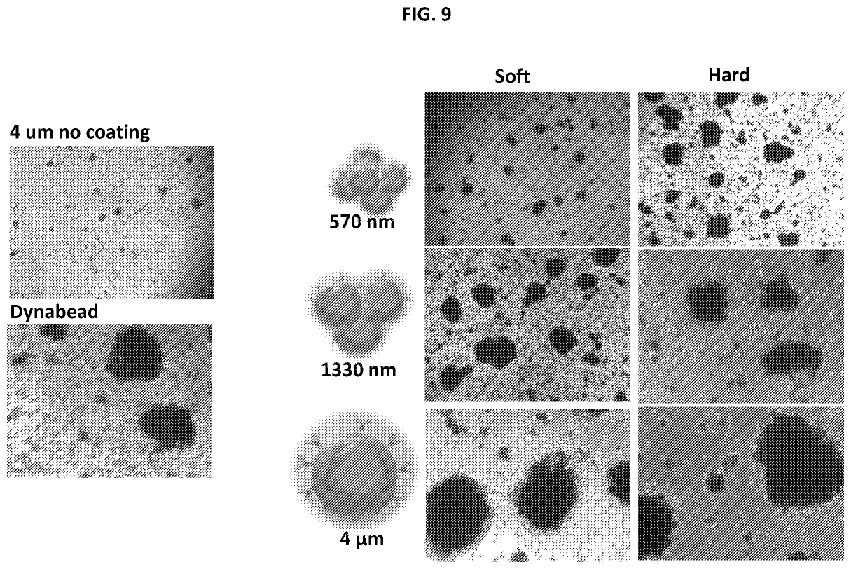

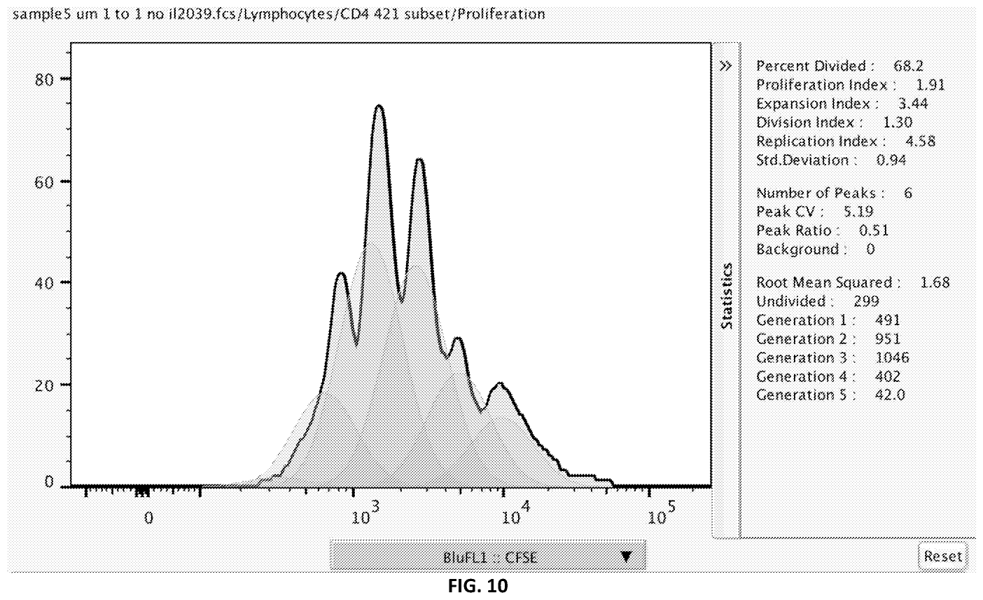

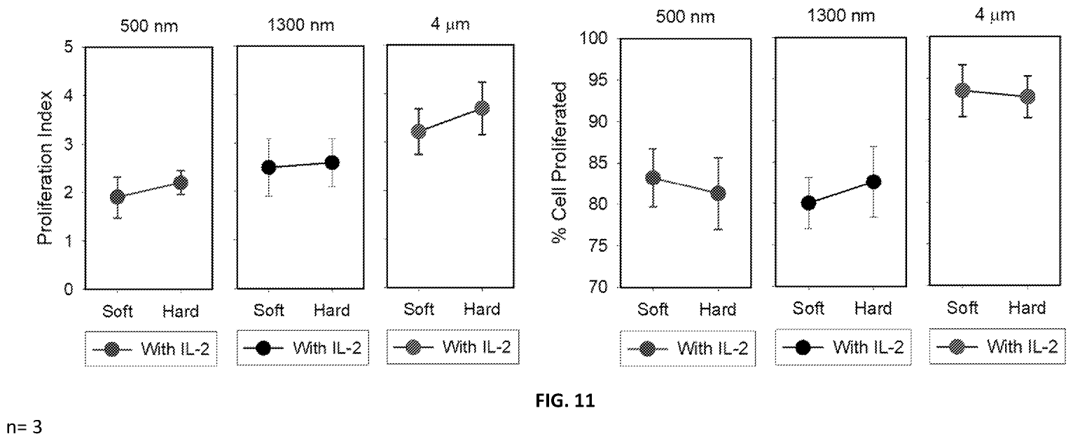

Three different sizes of particles were prepared (FIG. 9). The largest ones (4 microns (μm) diameter) provided the most stimulation, as seen by measurement of proliferation (FIGS. 10 and 11). FIG. 10 shows the method by which proliferation characteristics such as proliferation index and % proliferated cells are calculated, shown in FIG. 11. Hard and soft microparticles were prepared by varying the conditions as described above. The soft microparticles had a 3 kPa Young's modulus and the hard, 20 kPa.

T cells are optimized to perform specific effector functions in vivo. Activating T cells ex vivo to maximally elicit these responses is necessary to achieve the bulk numbers of cells needed to successfully fight solid tumors, and in general represents the mainstay approach for all engineered T cell therapies. The microparticles of the invention are provided to enhance targeting of tumors and activation of T cells against tumor antigens. Inspired by APCs such as B cells and DCs that present tumor neoantigens on MHC-II and express costimulatory ligands (e.g., B7-1 and B7-2), and tumors that themselves present neoantigens on MHC-I, in one embodiment, the microparticles of the present invention capture the membranes of these cells bearing antigen presenting and costimulatory capability.

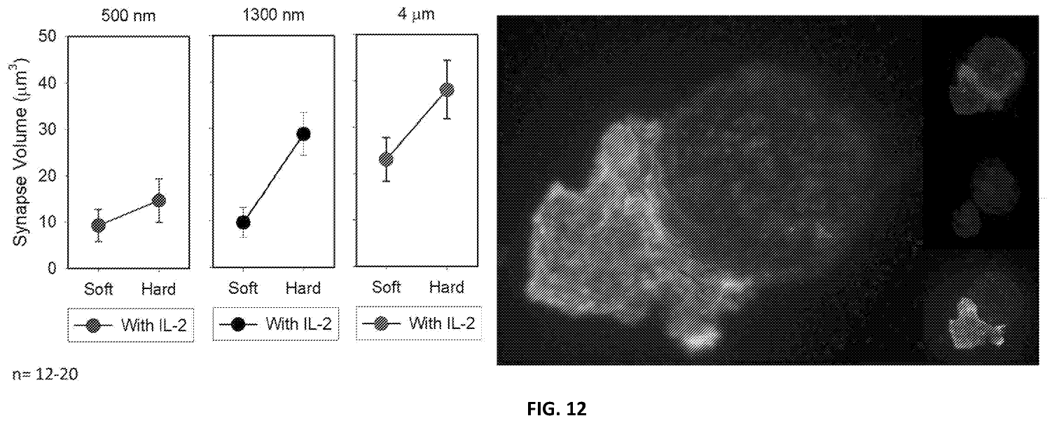

Furthermore, as shown in FIG. 12, the three sizes of microparticles formed immune synapses, but higher synapse volumes were achieved with the hard microparticles. The 4-5 μm microparticles were optimal for T cell activation.

To efficiently induce the formation of regulatory T cells (Tregs) from CD4+ cells, the microparticles of the invention should release TGF-β and IL-2. FIG. 15 depicts the potential release of from the microparticles described herein. TGF-β protein was loaded inside microparticles, and the interaction affinity, loading efficiency, and release over time (in PBS 37C under gentle shaking) were studied. The alginate-heparin MPs have a greater affinity for TGF-β and a higher binding efficiency. The cumulative TGF-β release from MPs is shown in FIG. 14. For the anti-cancer utilities of the MPs of the invention, induction of Tregs is undesirable; for potential use in the treatment of autoimmune disorders, such induction of Tregs is desirable. These embodiments of the invention are described hereinabove, wherein suppression of Treg induction can be achieved by including a TGF-β inhibitor in the microparticles of the invention; conversely, to enhance Treg induction, TGF-β can be included.

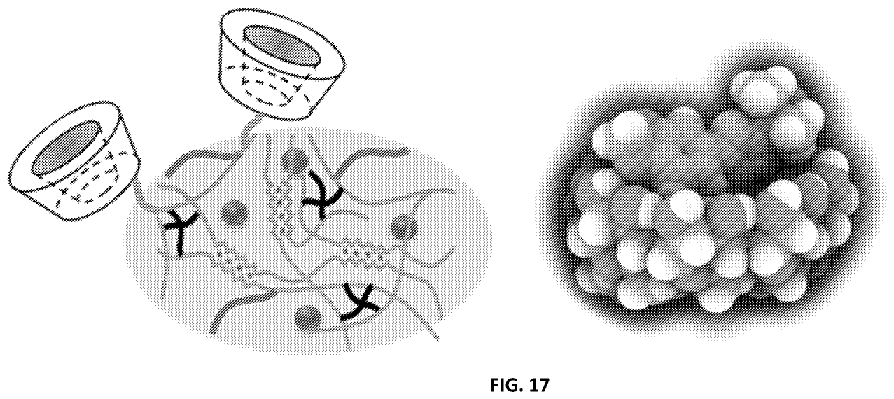

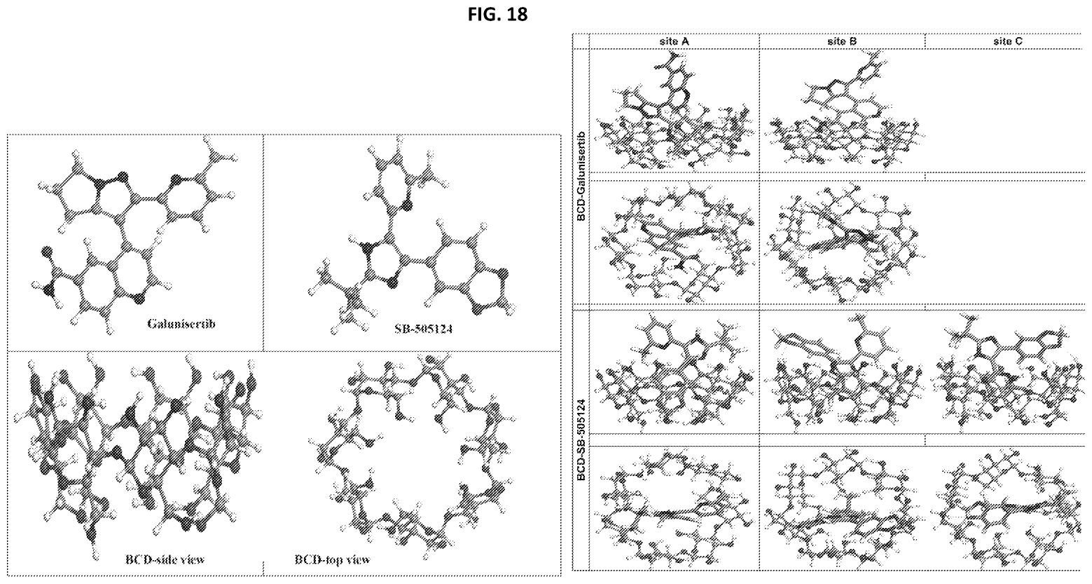

To efficiently stimulate the proliferation of cytotoxic T cells, the microparticles of the invention should suppress formation of regulatory T cells (Tregs) from CD4+ cells attracted to the microparticles. Sustained presence of TGF-β inhibitors can suppress Tregs formation. FIG. 15 and FIG. 16 depict two TGF-β inhibitors used here to show the suppression effect. Galunisertib showed higher degree of Tregs suppression as evaluated based on Foxp3 expression (FIG. 16). Incorporation of β-cyclodextrin was used to increase the affinity and loading of Galunisertib to microparticles (FIG. 17). β-cyclodextrin was conjugated to Alginate or Alginate-Heparin polymers. FIG. 18 depicted the strong affinity of Galunisertib towards β-cyclodextrin as assessed by molecular dynamic simulation.

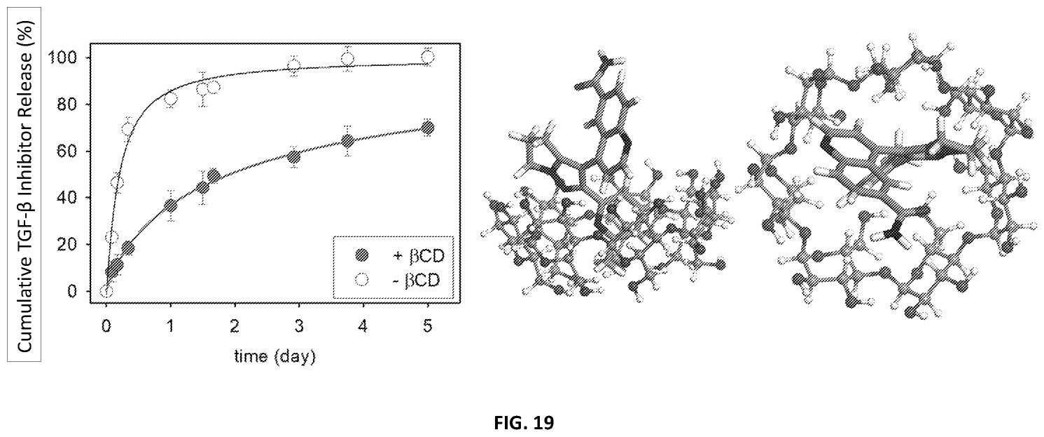

FIG. 19 depicts the potential release of TGF-β inhibitors from the microparticles described herein. TGF-βi molecules (here Galunisertib) was loaded inside microparticles, and the release over time (in PBS 37C under gentle shaking) was studied. The alginate-heparin MPs modified with β-cyclodextrin had a greater affinity for TGF-β inhibitor and a slower release. The cumulative TGF-βi release from microparticles (MPs) is shown. For the anti-cancer utilities of the MPs of the invention, induction of Tregs is undesirable; for potential use in the treatment of autoimmune disorders, such induction of Tregs is desirable.

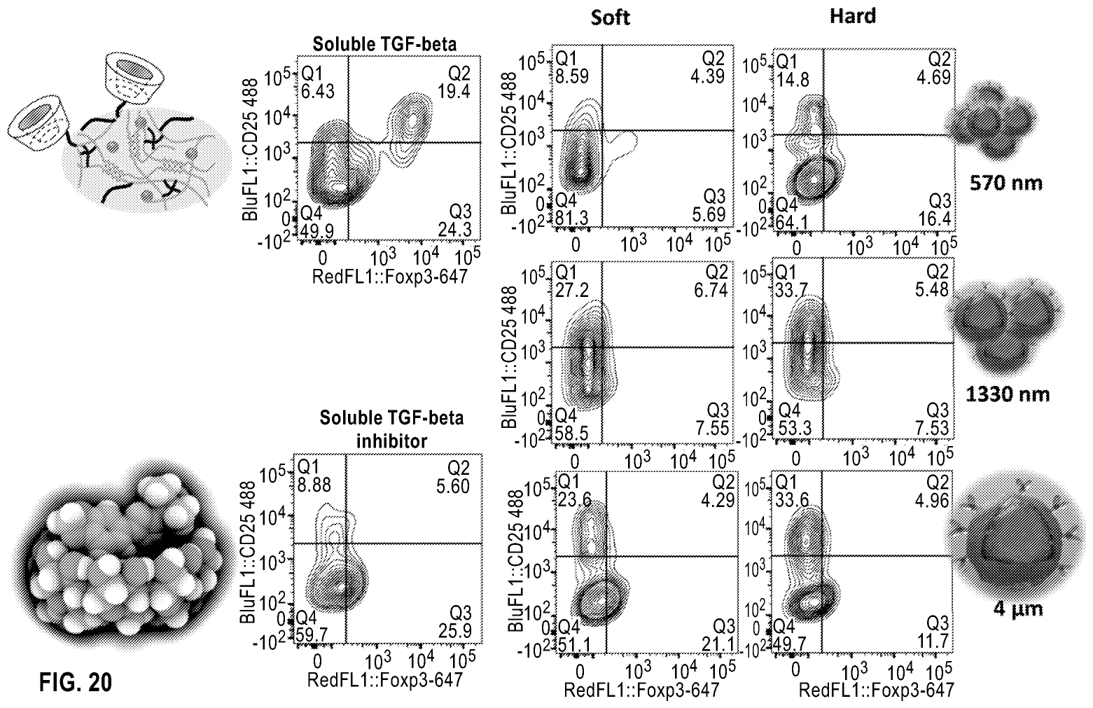

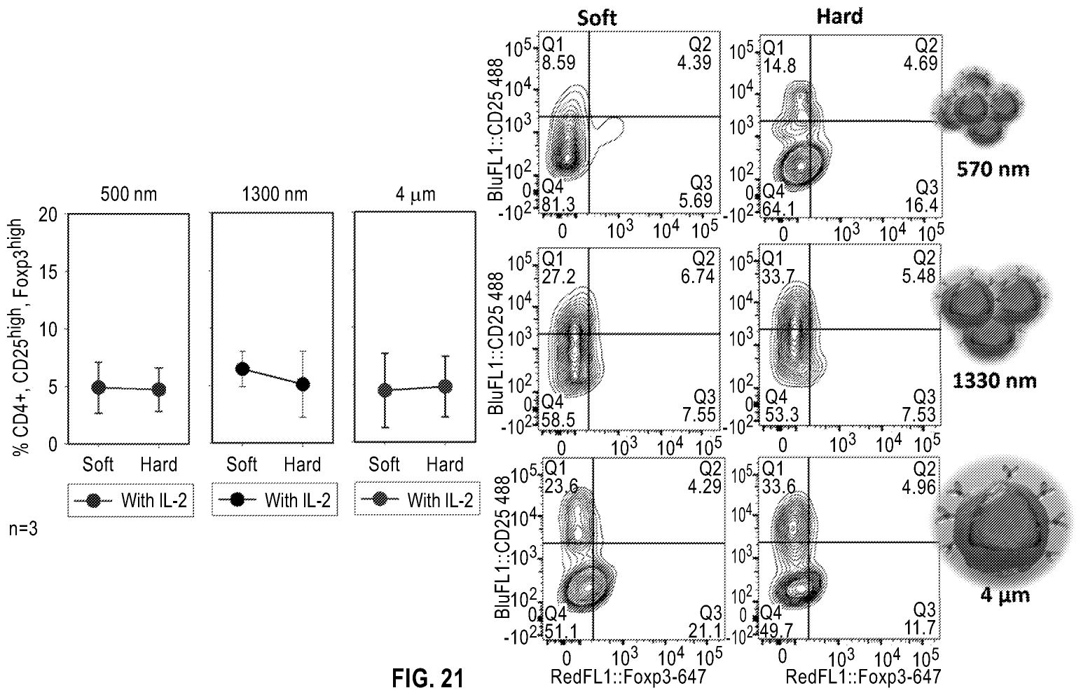

FIG. 20 and FIG. 21 depict inhibition of Tregs formation due to sustained release of TGF-β inhibitor from soft and stiff microparticles and nanoparticles at different sizes. Formation of Tregs was assessed by the expression of Foxp3 in activated (CD25+) CD4+ T cells.

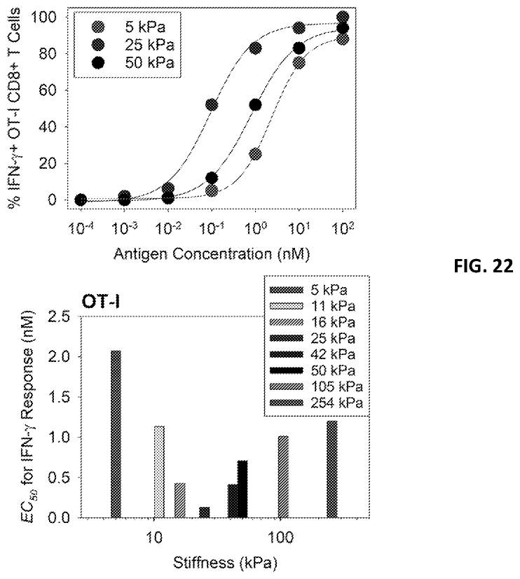

FIG. 22 depicts the effect of microparticle mechanical properties of B-cell membrane MPs treated with SIINFEKL (SEQ ID NO: 1) (ovalbumin peptide) and cultured with naïve OT-I T cells prepared by negative selection from mouse spleens ex vivo. The MPs were loaded with IL-2 as well. After 4 days, cells were tested using flow cytometry via intracellular cytokine assay (ICCS) to assess secretion of pro-inflammatory cytokines (here interferon-gamma [IFN-γ]). IFN-γ positive CD8+ T cells are known as effector (killer) T cells with advanced antitumor activity. Dependency of the activation to the antigen concentration was evaluated, and the concentration of an antigen that gives half-maximal T cell response (EC50) was evaluated to highlight the role of particle design on T cell response.

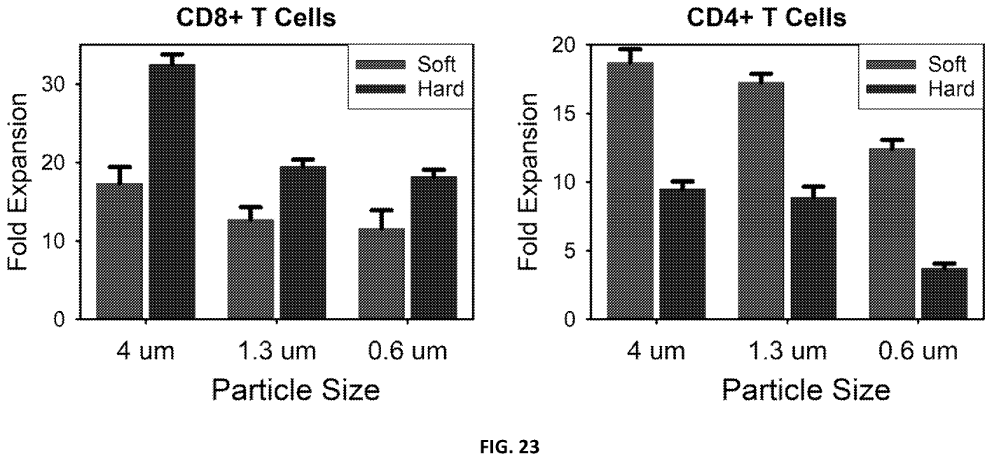

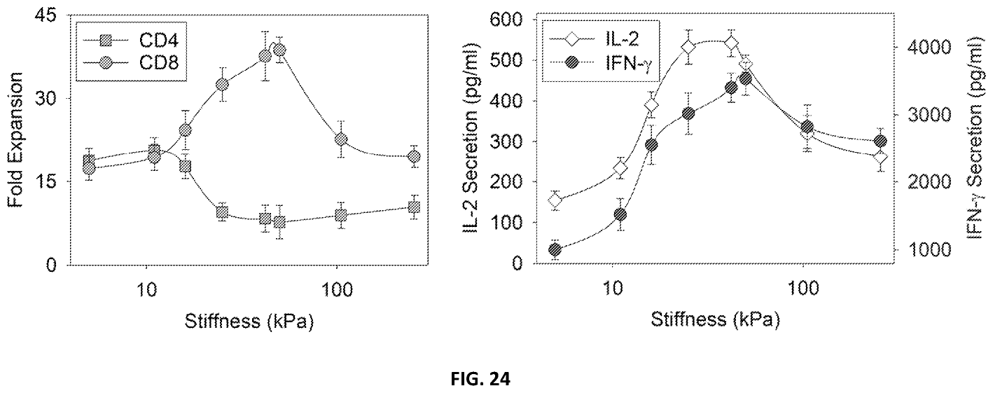

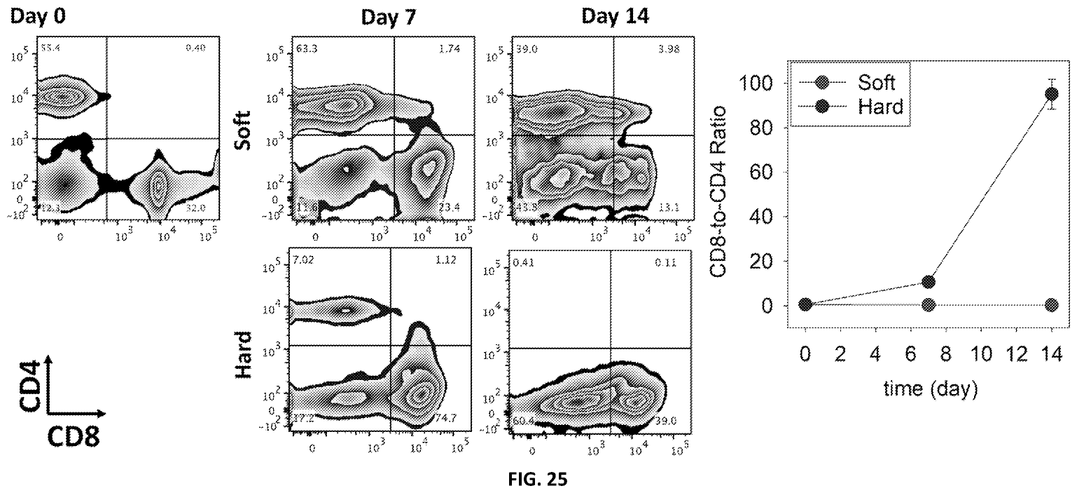

FIG. 23 and FIG. 24 depict the effect of microparticle mechanical properties of B-cell membrane MPs were either treated with SIINFEKL (SEQ ID NO: 1) (ovalbumin peptide) or ovalbumin peptide (323-339) for naïve OT-I (CD8+) or OR-II (CD8+) T cells, respectively, as prepared by negative selection from mouse spleens ex vivo. The MPs were loaded with IL-2 as well. The expansion of CD4+ and CD8+ T cells were evaluated as a function of particle size and mechanical properties. After 4 days, cells were tested using ELISA to assess secretion of inflammatory cytokines (here IL-2 and IFN-gamma [IFN-γ]). Dependency of the expansion and inflammatory cytokines secretions to the mechanical properties of the microparticles were evaluated as demonstrated in FIG. 24. FIG. 25 depicts how when microparticles were loaded with both ovalbumin peptides specific for transgenic CD4 and CD8 T cells, microparticles with stiff mechanical properties favored expansion of CD8+ T cells which have stronger tumor fighting properties compared to CD4+ T cells.

### Example 14

### Evaluation of T Cells Activated By Microparticles To Kill Tumor Cells & In Vivo Tumor Suppression Assay

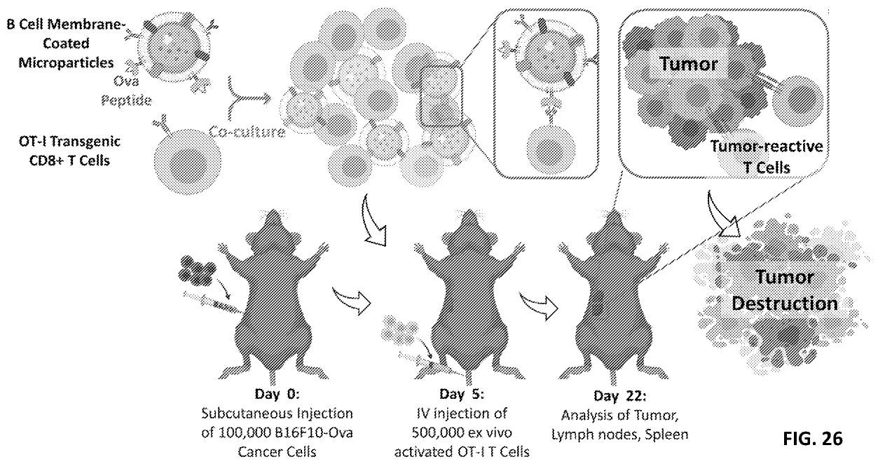

As shown in FIG. 26, 1×10⁵ (1×105) B16F10-OVA cells were subcutaneously injected into right flanks of C57BL/6J wild type (WT) mice (6-8 weeks old) mice (Weigelin, B. et al. Focusing and sustaining the antitumor CTL effector killer response by agonist anti-CD137 mAb. Proceedings of the National Academy of Sciences 112, 7551-7556 (2015)). These melanoma-derived cells are transfected to express chicken ovalbumin peptide (OVA) (Bellone, M. et al. Relevance of the tumor antigen in the validation of three vaccination strategies for melanoma. The Journal of Immunology 165, 2651-2656 (2000)). OT-I T cells were activated ex vivo with peptide-loaded cell membrane-coated microparticles for 4 days. 5×10⁵ (5×105) OT-I T cells were transferred intravenously on day 5 by retro-orbital injections (100 microliters [μL] per animal).

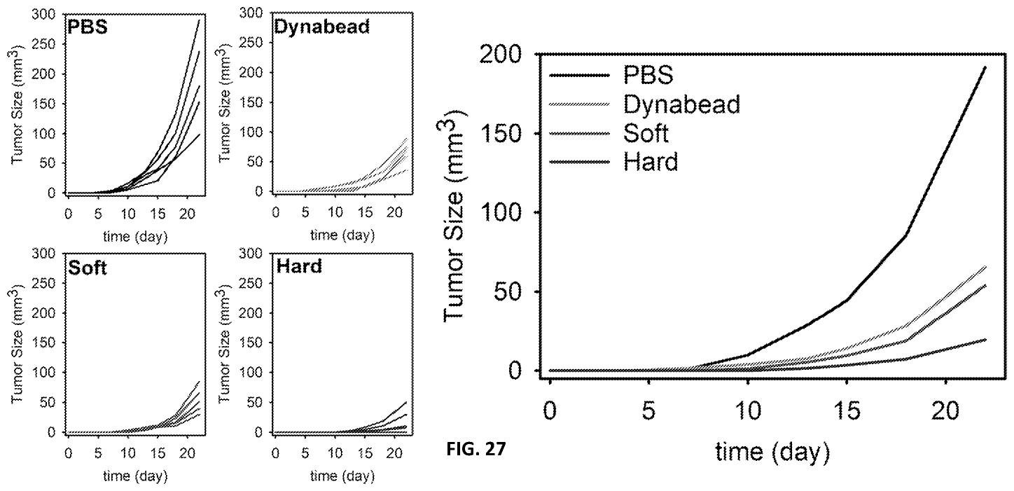

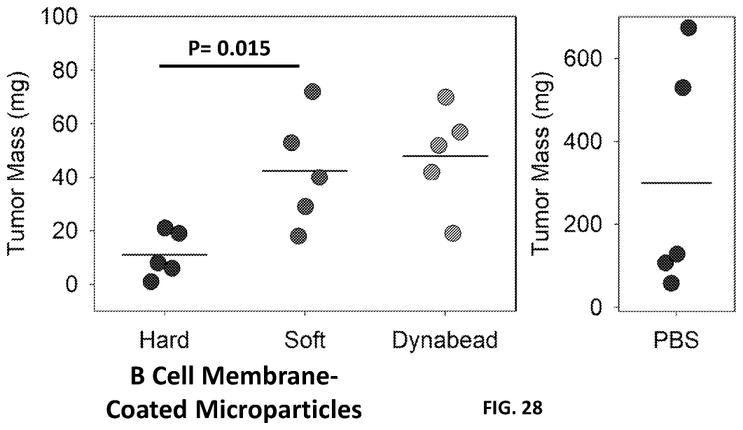

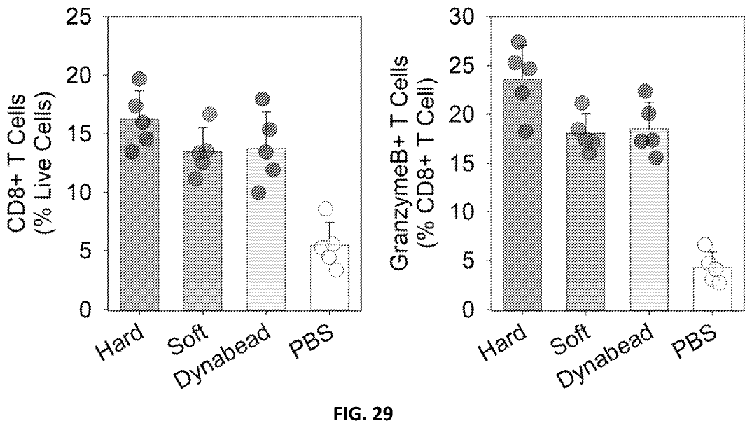

To test whether ex vivo activated T cells can fight solid tumors, B-cell membrane MPs were treated with SIINFEKL (SEQ ID NO: 1) (ovalbumin peptide) and cultured with naïve OT-I T cells prepared by negative selection from mouse spleens ex vivo (FIG. 27). The MPs were loaded with IL-2 as well. As a comparison, naive OT-I T cells were cultured in parallel with CD3-CD28 Dynabeads. After 2 days, cells were transferred away from the MPs or Dynabeads and into wells containing IL-2 (20 u/mL) for three more days. Mice were injected with B16 melanoma (100K cells) subcutaneously on day 0. On day 5, T cells were taken from culture, washed and transferred to PBS for injection, then injected intravenously. Tumor growth was followed over 22 days, as shown in FIG. 27. T cells activated by NPs cleared the tumors better than even DYNABEAD™-activated OT-Is. FIG. 28 depicts tumor masses measured upon sacrifice on day 22 for mice injected with OT-I T cells activated with SIINFEKL loaded B-cell membrane MPs. FIG. 29 is a series of graphs depicting the percentage of CD8+ T cells and granzyme B expressing CD8+ T cells in tumor T cells.

### Example 15

### Evaluation of T Cells Activated By Microparticles To Kill Tumor Cells

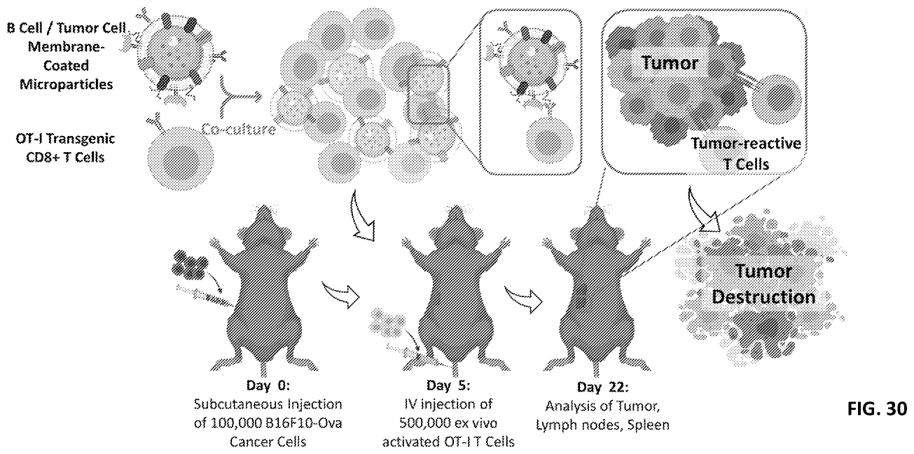

As shown in FIG. 30, 1×10⁵ (1×105) B16F10-OVA cells were subcutaneously injected into right left flanks of C57BL/6J wild type (WT) mice (6-8 weeks old) mice. OT-I T cells were activated ex vivo with either B cell membrane-coated or B cell/B16-F10-Ova melanoma cell membrane-coated microparticles (no peptide) for 4 days. 5×10⁵ (5×105) OT-I T cells were transferred intravenously on day 5 by retro-orbital injections (100 microliters [μL] per animal).

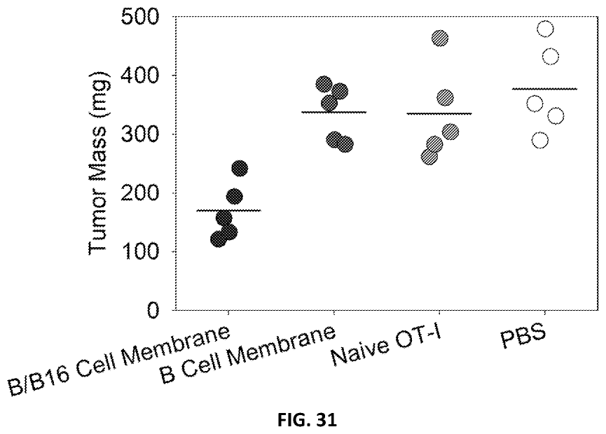

To test whether ex vivo activated T cells without presenting the ovalbumin peptide can fight solid tumors, B-cell or chimeric (B cell/B16-F10-Ova melanoma cell) membrane MPs were cultured with naïve OT-I T cells prepared by negative selection from mouse spleens ex vivo. The MPs were loaded with IL-2 as well. After 4 days, cells were transferred away from the MPs. Mice were injected with B16 melanoma (100K cells) subcutaneously on day 0. On day 5, T cells were taken from culture, washed and transferred to PBS for injection, then injected intravenously. Tumor growth was followed over 22 days. FIG. 31 shows the tumor masses measured upon sacrifice on day 22 for mice injected with OT-I T cells activated with mentioned cell membrane MPs.

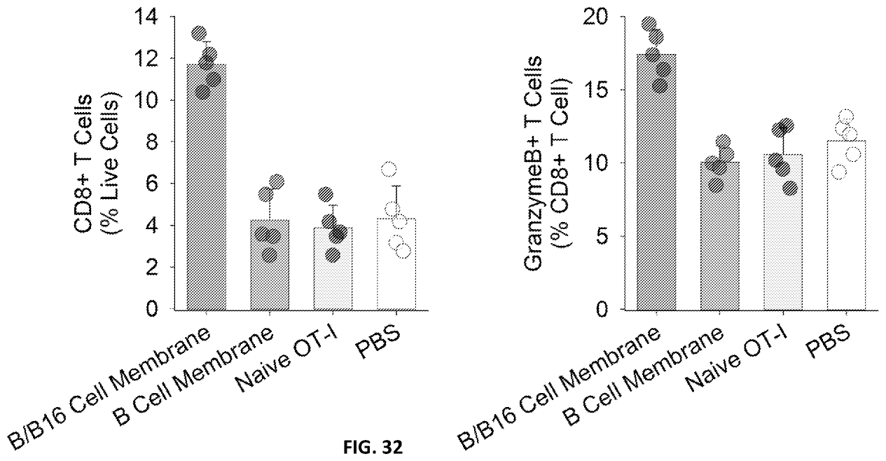

FIG. 32 is a series of graphs depicting the percentage of CD8+ T cells and granzyme B expressing CD8+ T cells in tumor T cells upon sacrifice of mice on day 22 for mice injected with OT-I T cells activated with mentioned cell membrane MPs.

### Example 16

### Evaluation of Microparticles To Train T Cells In Vivo And Kill Tumor Cells

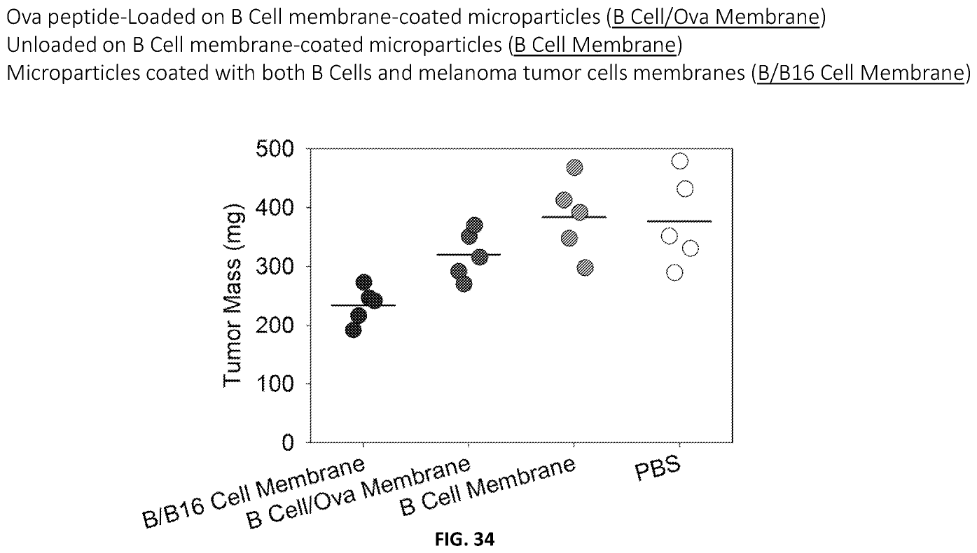

As shown in FIG. 34, 1×10⁵ (1×105) B16F10-OVA cells were subcutaneously injected into right flanks of C57BL/6J wild type (WT) mice (6-8 weeks old) mice. There was no administration of transgenic OT-I T cells here. B cell membrane-coated microparticles (with or without peptide) or B cell/B16-F10-Ova melanoma cell membrane-coated microparticles were injected subcutaneously close to tumor site on day 5.

To test whether in vivo activation of T cells to fight solid tumors, 1×10⁶ (1×106) MPs were subcutaneously injected close to tumor site on day 5. As there was no OT-I T cell transfer, the antitumoral response was relying on activation of endogenous T cells.

FIG. 34 shows the tumor masses measured upon sacrifice on day 22 for mice injected with cell membrane MPs.

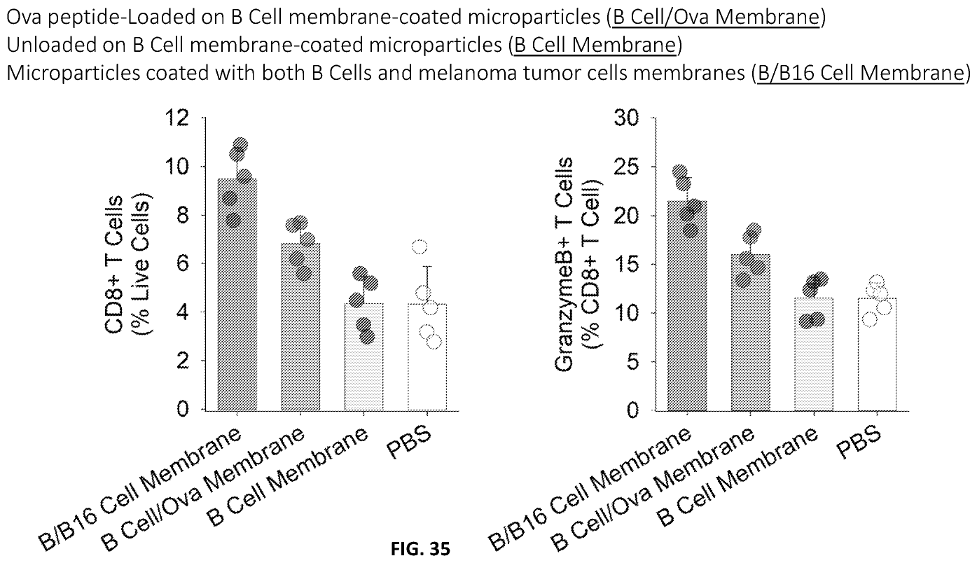

FIG. 35 is a series of graphs depicting the percentage of CD8+ T cells and granzyme B expressing CD8+ T cells in tumor T cells upon sacrifice of mice on day 22 for mice injected with mentioned cell membrane MPs.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A microparticle comprising:
(a) an antigen specific to a cell or virus of interest; and
(b) a costimulatory component derived from an antigen presenting cell.

2. The microparticle of claim 1, wherein the antigen comprises a tumor antigen; a cancer-specific antigen; a self antigen; an IgE receptor-specific antigen; a pathogen-specific antigen; an antigen specific to an organ, tissue, or cell of interest; an antigen specific to a transplanted organ, tissue, or cell of interest; a macrophage-specific antigen; an erythrocyte-specific antigen; a platelet-specific antigen; a platelet factor 5-specific antigen; a fibrinogen-specific antigen; a stem cell- or progenitor cell-specific antigen; a lymphocyte-specific antigen; a monocyte-specific antigen; an immune cell-specific antigen; or a stromal cell-specific antigen or a combination thereof.

3. The microparticle of claim 2, wherein:
(a) the antigen comprises a tumor antigen comprising a membrane isolated from a tumor cell or a tumor tissue; or
(b) the antigen comprises a cancer-specific antigen comprising a membrane isolated from a cancer cell or a cancer tissue.

4. The microparticle of claim 2, wherein:
(a) the antigen comprises a self antigen comprising a membrane isolated from a cell, a tissue, or an organ targeted by an autoimmune disease or undergoing autoimmune attack;
(b) the antigen comprises an IgE receptor-specific antigen comprising a membrane isolated from a mast cell or a basophil in response to an allergic reaction or hypersensitivity reaction;
(c) the antigen comprises a pathogen-specific antigen comprising a membrane isolated from a cell of a pathogen or a viral envelope or a viral capsid;
(d) the antigen comprises a macrophage-specific antigen or an immune cell-specific antigen comprising a membrane isolated from a macrophage or an immune cell of interest;
(e) the antigen comprises an antigen specific to an organ, tissue, or cell of interest comprising a membrane isolated from the organ, tissue, or cell of interest;
(f) the antigen comprises an antigen specific to a transplanted organ, tissue, or cell of interest comprising a membrane isolated from the organ, tissue, or cell of interest;
(g) the antigen comprises an erythrocyte-specific antigen comprising a membrane isolated from an erythrocyte;
(h) a platelet-specific antigen, a platelet factor 5-specific antigen, a fibrinogen-specific antigen comprising a membrane isolated from a platelet;
(i) the antigen comprises a stem cell- or progenitor cell-specific antigen comprising a membrane isolated from a stem cell or a progenitor cell, respectively, of interest;
(j) a lymphocyte-specific antigen comprising a membrane isolated from a lymphocyte of interest;
(k) a monocyte-specific antigen comprising a membrane isolated from a monocyte (e.g., a leukocyte) of interest; or
(l) a stromal cell-specific antigen comprising a membrane isolated from a stromal cell.

5. The microparticle of claim 4, wherein the antigen comprises a B cell-specific antigen comprising a membrane isolated from a B cell or wherein the antigen comprises a T cell-specific antigen comprising a membrane isolated from a T cell.

6. The microparticle of claim 1, wherein the tumor cell or tumor tissue is obtained from a surgically obtained tumor, tumor biopsy, or tumor sample.

7. The microparticle of claim 6, wherein the cell, tissue, or organ of interest is obtained from a surgically obtained cell, tissue, or organ of interest, a biopsy, or a sample.

8. The microparticle of claim 1, wherein the antigen is obtained from a cell grown in vitro or a culture media thereof.

9. The microparticle of claim 1, wherein the costimulatory component from an antigen presenting cell comprises a membrane isolated from an antigen presenting cell.

10. The microparticle of claim 9, wherein the antigen presenting cell is stimulated in vitro before the membrane is isolated.

11. The microparticle of claim 1, wherein the microparticle comprises a polymer.

12. The microparticle of claim 11, wherein the polymer is a biocompatible polymer.

13. The microparticle of claim 12, wherein the polymer is alginate, hyaluronic acid, or chitosan.

14. The microparticle of claim 1, wherein the microparticle further comprises heparin.

15. The microparticle of claim 1, the microparticle further comprising alginate and heparin.

16. The microparticle of claim 1, wherein the polymer is cross-linked.

17. The microparticle of claim 1, further comprising paramagnetic nanoparticles.

18. The microparticle of claim 17, the paramagnetic nanoparticles comprising superparamagnetic iron oxide nanoparticles (SPIONs).

19. The microparticle of claim 1, further comprising at least one immunoregulatory compounds.

20. The microparticle of claim 19, wherein the at least one immunoregulatory compound comprises an immunostimulatory compound.

21. The microparticle of claim 19, wherein the at least one immunoregulatory compound comprises an immunosuppression compound.

22. The microparticle of claim 19, wherein the at least one immunoregulatory compound comprises a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid.

23. The microparticle of claim 22, wherein the cytokine comprises an interleukin (IL).

24. The microparticle of claim 23, wherein the interleukin comprises interleukin-2 (IL-2), interleukin-4 (IL-4), interleukin-6 (IL-6), interleukin-7 (IL-7), interleukin-10 (IL-10), interleukin-12 (IL-12), or interleukin-15 (IL-15) or an IL-2 superkine.

25. The microparticle of claim 24, wherein the IL-2 superkine comprises the sequence as set forth in SEQ ID NO: 3.

26. The microparticle of claim 22, wherein the chemokine comprises stromal cell-derived factor 1a (SDF-1a).

27. The microparticle of claim 22, wherein the growth factor comprises transforming growth factor-beta (TGF-β), vascular endothelial growth factor (VEGF), or bone morphogenetic protein-2 (BMP-2).

28. The microparticle of claim 22, wherein the microparticle comprises IL-2 and TGF-β.

29. The microparticle of any one of claims 1-28, further comprising at least one compound that regulates induction of regulatory T cells (Tregs).

30. The microparticle of claim 29, wherein the at least one compound that regulates induction of Tregs comprises a compound that suppresses induction of Tregs.

31. The microparticle of claim 30, wherein the compound that suppresses induction of Tregs comprises a TGF-β inhibitor.

32. The microparticle of claim 31, wherein the TGF-β inhibitor is a TGF-β receptor inhibitor.

33. The microparticle of claim 31, wherein the TGF-β inhibitor is galinusertib (LY2157299) or SB505124.

34. The microparticle of claim 29, wherein the at least one compound that regulates induction of Tregs comprises a compound that induces Tregs.

35. The microparticle of claim 34, wherein the compound that induces Tregs is a TGF-β or an activator thereof.

36. The microparticle of claim 1, further comprising a small molecule.

37. The microparticle of claim 1, further comprising a hydrophobic therapeutic agent.

38. The microparticle of claim 1, wherein:
(a) the antigen comprises a tumor antigen; and
(b) the microparticle further comprises at least one immunoregulatory compound comprising a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid.

39. The microparticle of claim 38, wherein:
(a) the cytokine comprises IL-2, IL-4, IL-6, IL-7, IL-10, IL-12, IL-15, or an IL-2 superkine;
(b) the chemokine comprises SDF-1a; or (c) the growth factor comprises TGF-β, VEGF, or BMP-2.

40. The microparticle of claim 38, further comprising a TGF-β inhibitor.

41. The microparticle of claim 38, wherein the tumor antigen is specific for a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1 ;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

42. The microparticle of claim 1, wherein:
(a) the antigen comprises a self antigen; and
(b) the microparticle further comprises at least one immunoregulatory compound comprising a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid.

43. The microparticle of claim 42, wherein:
(a) the cytokine comprises IL-2, IL-4, IL-6, IL-7, IL-10, IL-12, or IL-15 or an IL-2 superkine;
(b) the chemokine comprises SDF-1a; or
(c) the growth factor comprises TGF-β, VEGF, or BMP-2.

44. The microparticle of claim 42, further comprising IL-2, TGF-β, or a TGF-β activator.

45. A nanoparticle comprising:
(a) an antigen specific to a cell or virus of interest; and
(b) a costimulatory component derived from an antigen presenting cell.

46. A method for preparing an immunoactive microparticle or an immunoactive nanoparticle, the method comprising:
(a) providing a biocompatible polymer;
(b) combining said biocompatible polymer in a microfluidic droplet generator with a crosslinker to form the microparticle or the nanoparticle;
(c) incubating the microparticle or the nanoparticle with:
(i) an antigen specific to a cell or virus of interest; and
(ii) a costimulatory component derived from an antigen presenting cell.

47. A method for preparing an activated cytotoxic T cell population specific for an antigen of interest comprising:
(a) providing a microparticle or a nanoparticle comprising:
(i) an antigen specific to a cell or virus of interest; and
(ii) a costimulatory component derived from an antigen presenting cell;
(b) obtaining leukocytes from a subject; and
(c) exposing the leukocytes in vitro or ex vivo to the microparticle or the nanoparticle.

48. The method of claim 47, wherein the antigen comprises membranes isolated from cells or tissues obtained from the subject.

49. The method of claim 47, wherein the leukocytes are obtained from whole blood or pheresis.

50. The method of claim 47, wherein the microparticle further comprises paramagnetic nanoparticles and is separated from the leukocytes by magnetic separation.

51. The method of claim 47, wherein the antigen comprises a tumor antigen; a cancer-specific antigen; a self antigen; an IgE receptor-specific antigen; a pathogen-specific antigen; an antigen specific to an organ, tissue, or cell of interest; an antigen specific to a transplanted organ, tissue, or cell of interest; a macrophage-specific antigen; an erythrocyte-specific antigen; a platelet-specific antigen; a platelet factor 5-specific antigen; a fibrinogen-specific antigen; a stem cell- or progenitor cell-specific antigen; a lymphocyte-specific antigen; a monocyte-specific antigen; an immune cell-specific antigen; or a stromal cell-specific antigen; or a combination thereof.

52. The method of claim 51, wherein:
(a) the antigen comprises a tumor antigen comprising a membrane isolated from a tumor cell or a tumor tissue;
(b) the antigen comprises a cancer-specific antigen comprising a membrane isolated from a cancer cell or a cancer tissue;
(c) the antigen comprises a self antigen comprising a membrane isolated from a cell, a tissue, or an organ targeted by an autoimmune disease or undergoing autoimmune attack;
(d) the antigen comprises an IgE receptor-specific antigen comprising a membrane isolated from a mast cell or a basophil in response to an allergic reaction or hypersensitivity reaction;
(e) the antigen comprises a pathogen-specific antigen comprising a membrane isolated from a cell of a pathogen or a viral envelope or a viral capsid;
(f) the antigen comprises a macrophage-specific antigen or an immune cell-specific antigen comprising a membrane isolated from a macrophage or an immune cell of interest;
(g) the antigen comprises an antigen specific to an organ, tissue, or cell of interest comprising a membrane isolated from the organ, tissue, or cell of interest;
(h) the antigen comprises an antigen specific to a transplanted organ, tissue, or cell of interest comprising a membrane isolated from the organ, tissue, or cell of interest;
(i) the antigen comprises an erythrocyte-specific antigen comprising a membrane isolated from an erythrocyte;
(j) the antigen comprises a platelet-specific antigen, a platelet factor 5-specific antigen, a fibrinogen-specific antigen comprising a membrane isolated from a platelet;
(k) the antigen comprises a stem cell- or progenitor cell-specific antigen comprising a membrane isolated from a stem cell or a progenitor cell, respectively, of interest;
(l) a lymphocyte-specific antigen comprising a membrane isolated from a lymphocyte of interest;
(m) a monocyte-specific antigen comprising a membrane isolated from a monocyte (e.g., a leukocyte) of interest; or
(n) a stromal cell-specific antigen comprising a membrane isolated from a stromal cell.

53. The method of claim 52, wherein the antigen comprises a B cell-specific antigen comprising a membrane isolated from a B cell of interest, or a T cell-specific antigen comprising a membrane isolated from a T cell of interest.

54. The method of claim 47, wherein the microparticle further comprises at least one immunoregulatory compound, wherein the at least one immunoregulatory compound comprises a cytokine, a growth factor, a chemokine, a therapeutic or diagnostic antibody or fragment thereof, an antigen-binding protein, a Fc fusion protein, an anticoagulant, an enzyme, a hormone, a thrombolytic, a peptide, an oligonucleotide, or a nucleic acid.

55. The method of claim 54, wherein the cytokine comprises an interleukin (IL).

56. The method of claim 55, wherein the interleukin comprises IL-2, IL-4, IL-6, IL-7, IL-10, IL-12, IL-15, or an IL-2 superkine.

57. The method of claim 56, wherein the IL-2 superkine comprises the sequence as set forth in SEQ ID NO: 3.

58. The method of claim 47, wherein the microparticle further comprises at least one compound that regulates induction of regulatory T cells (Tregs).

59. The method of claim 58, wherein the at least one compound that regulates induction of Tregs comprises a compound that suppresses induction of Tregs.

60. The method of claim 59, wherein the compound that suppresses induction of Tregs comprises a TGF-β inhibitor.

61. The method of claim 60, wherein the TGF-β inhibitor is a TGF-β receptor inhibitor.

62. The method of claim 60, wherein the TGF-β inhibitor is galinusertib (LY2157299) or SB505124.

63. The method of claim 58, wherein the at least one compound that regulates induction of Tregs comprises a compound that induces Tregs.

64. The method of claim 63, wherein the compound that induces Tregs is a TGF-β or an activator thereof.

65. A method for treating a disease or medical condition, or of alleviating symptoms thereof, at a focus of interest in a subject in need, said method comprising:
(a) providing a microparticle or a nanoparticle comprising:
(i) an antigen specific to a cell or virus of interest; and
(ii) a costimulatory component derived from an antigen presenting cell;
(b) obtaining a leukocyte from the subject;
(c) exposing the leukocyte to the microparticle or the nanoparticle; and
(d) infusing the leukocytes into the subject.

66. The method of claim 65, wherein the antigen is obtained from a sample from the subject.

67. The method of claim 65, wherein the leukocyte is obtained from whole blood or pheresis.

68. The method of claim 65, wherein the microparticle further comprises paramagnetic nanoparticles and is separated from the leukocyte by magnetic separation.

69. The method of claim 65, wherein the subject is treated with at least one other immunotherapy.

70. The method of claim 65, wherein:
(a) the disease or medical condition comprises a tumor, a suspected tumor, or a resected tumor;
(b) the disease or medical condition comprises an autoimmune disease;
(c) the disease or medical condition comprises an allergic reaction or hypersensitivity reaction;
(d) the disease or medical condition comprises a localized infection or an infectious disease;
(e) the disease or medical condition comprises an injury or a site of chronic damage;
(f) the disease or medical condition comprises a surgical site;
(g) the disease or medical condition comprises a transplanted organ, tissue, or cell; or
(h) the disease or medical condition comprises a blood clot causing or at risk for causing a myocardial infarction, an ischemic stroke, or a pulmonary embolism.

71. The method of claim 70, said tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/ peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

72. The method of claim 65, wherein said treating:
(a) reduces the size of the tumor, eliminates said tumor, slows the growth or regrowth of the tumor, or prolongs survival of said subject, or any combination thereof.
(b) reduces or eliminates inflammation or another symptom of said autoimmune-targeted or symptomatic focus of said autoimmune disease, prolongs survival of said subject, or any combination thereof;
(c) reduces or eliminates inflammation or another symptom of allergic reaction or hypersensitivity reaction at said reactive focus of said allergic reaction or hypersensitivity reaction, prolongs survival of said subject, or any combination thereof;
(d) reduces or eliminates infection or symptoms at said focus of infection or symptoms of said localized infection or infectious disease, prolongs survival of said subject, or any combination thereof;
(e) reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof;
(f) reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof;
(g) reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or
(h) reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof.

73. The method of claim 65, wherein at the site, T cells are stimulated to target the antigen, and the induction of Tregs is suppressed.

74. The method of claim 65, wherein at the site, T cells are suppressed from targeting the antigen, and Tregs are induced.

75. The method of claim 65, wherein the disease or medical condition comprises a tumor or a cancer, the antigen comprises a tumor antigen or a cancer antigen, and the subject is treated with at least one other anti-tumor or anti-cancer therapy.

76. The method of claim 75, wherein the tumor is a solid tumor.

77. The method of claim 75, wherein the tumor is an inoperable tumor.

78. The method of claim 75, wherein the tumor comprises a cancerous tumor, a pre-cancerous tumor, or a non-cancerous tumor.

79. The method of claim 65, wherein the disease or medical condition comprises an autoimmune disease, the antigen comprises a self antigen, and the subject is treated with at least one other immunotherapy.


SEQUENCE LISTING (3 sequences)

SEQ ID NO: 1 - protein, 8 aa, Artificial
  Synthetic
SIINFEKL

SEQ ID NO: 2 - DNA, 420 bp, Artificial
  Synthetic
ggagccatgggagaattcgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcagagcaaaaactttcacttcgatcccagggacgtcgtcagcaatatcaacgtattcgtcctggaactaaagggatctgaaacaacattcatgtgtgaatatgctgatgagacagcaaccattgtagaatttctgaacagatggattaccttttgtcaaagcatcatctcaacactaactcat

SEQ ID NO: 3 - protein, 138 aa, Artificial
  Synthetic
MGEFAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTH